(19) United States
(12) Patent Application Publication
(10) Pub. No.: US 2008/0057122 A1
(43) Pub. Date: Mar. 6, 2008

(54) Acetaminophen pharmaceutical compositions

(75) Inventors: Tania E. Toney-Parker, Hampstead, NC (US); Jack Lawrence James, Castle Hayne, NC (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: aaiPharma Inc., Wilmington, NC (US)

(21) Appl. No.: 11/513,906

(22) Filed: Aug. 31, 2006

(51) Int. Cl. A61K 31/485; A61K 9/22

(52) U.S. Cl. 424/468; 514/282

(57) ABSTRACT

Modified release dosage forms for daily oral dosing to a human patient for providing relief from pain are provided. The modified release dosage form comprises an immediate release component and a sustained release component, wherein the immediate release component and the sustained release component collectively contain a therapeutically effective amount of acetaminophen and an optional therapeutically effective amount of active pharmaceutical ingredient such as an opioid or opioid-like analgesic. In one embodiment, the opioid or opioid-like analgesic is propoxyphene napsylate or propoxyphene HCl salt.

### FIELD OF THE INVENTION

The present invention relates to acetaminophen modified release pharmaceutical dosage forms for administration to a subject, particularly humans, in need of treatment for pain. The acetaminophen dosage form comprises an immediate release component and an extended release component wherein, when said dosage form is administered to a subject, said formulation provides eight-hour bioequivalence of acetaminophen compared to two consecutive administrations of one-half equivalent dose concentrations of acetaminophen at a four-hour interval. The present invention also relates to a method for treating pain using the pharmaceutical dosage form as well as a method of making the pharmaceutical dosage form.

### BACKGROUND OF PRESENT INVENTION

Acetaminophen (APAP) is a well-known analgesic and antipyretic drug. In the United States, it is available for non-prescription over-the-counter sale in conventional liquid, suppository, capsule, tablet and caplet dosage forms. The tablet and caplet dosage forms typically contain 325 mg APAP as “regular strength” or 650 mg as “extra strength”Normally, regular strength tablets or caplets are taken as one or two every four to six hours, and the extra strength tablets or caplets are taken as one or two every six hours. It would be desirable to extend the dosing interval while maintaining the initial plasma concentrations achievable with conventional tablets or caplets. This would provide immediate and extended therapeutic analgesic or antipyretic effect and reduce the number of doses necessary, thereby making therapy more convenient and potentially improve patient compliance. Based on a recent study on the effects of high doses of acetaminophen on the liver, the American Liver Foundation now recommends that the total daily amounts of acetaminophen not exceed three grams for any prolonged period of time. See, for instance, the American Liver Foundation press release dated Jul. 18, 2006. URL:http://www.liverfoundation.org/db/pressrelease/71 A way to do this has now been found, using the present invention, wherein when the inventive pharmaceutical composition is administered to a subject, the composition provides eight-hour bioequivalence of APAP compared to two consecutive administrations of one-half equivalent does concentrations of APAP at a four-hour interval, thus reducing the total number of doses needed per day. One or more optional API(s) ingredients such as opioids or opioid-like compounds may be included in the pharmaceutical composition to provide an additional therapeutic effect.

### SUMMARY OF THE INVENTION

The present invention relates to pharmaceutical formulations that provides at least eight-hour bioequivalence of APAP, methods of treatment using the pharmaceutical formulations and method for preparing the pharmaceutical formulations.

In one embodiment of the invention, a pharmaceutical formulation is disclosed which comprises an immediate release component and an extended release component wherein, when said formulation is administered to a subject, said formulation provides eight-hour bioequivalence of APAP compared to two consecutive administrations of one-half equivalent dose concentrations of APAP at a four-hour interval.

In one aspect of this embodiment, at least one active pharmaceutical ingredient (API) is added to at least one of said immediate release component or said extended release component or both. Representative examples include, for instance, an opioid or opioid-like compound, a non-steroidal anti-inflammatory drug (NSAID), a cyclooxygenase-II (COX-2) inhibitor, a glycine receptor antagonist, an antitussive, an expectorant, a decongestant, an antihistamine and mixtures thereof. Representative examples of opioid or opiod-like compounds include propoxyphene salt such as propoxyphene napsylate or hydrochloride salts, hydrocodone, or oxycodone.

In another aspect of this embodiment, the pharmaceutical formulation further has a 99.9% confidence interval for which the formulation, when administered to a subject in a single dose and at a total dosage strength of 1300 mg APAP under fasting conditions, the maximum plasma concentration (Cmax) of the APAP following administration is in the range from about 5.89_ug/mL to about 9.52_ug/mL.

In another embodiment of the invention, a pharmaceutical formulation is provided which comprises an immediate release component and an extended release component wherein at least one API is added to the immediate release component, the extended release component or both, and wherein when said formulation is administered to a subject, said formulation provides eight-hour bioequivalence of APAP compared to two consecutive administrations of one-half equivalent dose concentrations of APAP at a four-hour interval.

In one aspect of this embodiment, the pharmaceutical formulation includes propoxyphene napsylate or hydrochloride salt and further has a 99.9% confidence interval for which the formulation, when administered to a subject in a single dose and at a total dosage strength of 1300 mg APAP and 200 mg proxyphene napsylate under fasting conditions, the maximum plasma concentration (Cmax) of the APAP following administration is in the range from about 5.89_ug/mL to about 9.52_ug/mL and the Cmax for propoxyphene napsylate is in the range from about 56 to about 98 ng/mL/mg

In another embodiment of the invention, a pharmaceutical formulation is provided in unit dosage form comprising per dosage unit an amount of APAP within a range from about 325 to about 1300 mg of a composition comprising an immediate release component and an extended release component wherein, when said formulation is administered to a subject, said formulation provides eight-hour bioequivalence of APAP compared to two consecutive administrations of one-half equivalent dose concentrations of APAP at a four-hour interval, and wherein said formulation in unit dosage form being adapted for oral administration.

In another embodiment of the invention, the pharmaceutical formulation comprises a first active ingredient which is APAP and an optional second active ingredient selected from the group consisting of one or more opiod or opiod-like compound wherein, when said formulation is administered to a subject in a single administration and at a total dosage strength of 1300 mg APAP under fasting conditions, the maximum plasma concentration (Cmax) of the APAP following administration is in the range from about 5.89_ug/mL to about 9.52_ug/mL.

In another embodiment of the invention, a pharmaceutical formulation is provided which comprises a blend of (a) an immediate release component comprising acetaminophen, an optional water soluble binder, and at least one disintegrant; and (b) an extended release component comprising at least one controlled release matrix polymer, and one or more optional fillers, and one or more optional wicking agents.

In yet another embodiment of the invention, a pharmaceutical formulation is provided which is prepared by the process comprising the steps of: (a) blending acetaminophen, a water soluble binder, a disintegrant, and one or more optional API(s) to form an immediate release component; (b) granulating and milling a composition comprising at least one controlled release matrix polymer to form an extended release component; and (c) blending the immediate release component, the extended release component, one or more optional binders, one or more optional glidants, and one or more optional lubricants to form the pharmaceutical composition.

In yet another embodiment of the invention, a pharmaceutical formulation is provided which comprises a blend of an immediate release component comprising APAP and an extended release component comprising optional APAP wherein when said formulation is administered to a subject for the treatment of pain for at least 8 hours, providing said formulation is not in the form of a bi-layer tablet. In another embodiment of the invention, a method is provided for treating pain in a subject in need of such treatment comprising administering a pharmaceutically effective amount of a pharmaceutical formulation of the invention.

In another embodiment of the invention, a method is provided for treating pain and a disease state resulting from elevated histamine levels in a subject in need of such treatment comprising administering a pharmaceutically effective amount of a pharmaceutical formulation comprising an immediate release component, an extended release component, APAP and an antihistimine wherein, when said formulation is administered to a subject, said formulation provides eight-hour bioequivalence of APAP compared to two consecutive administrations of one-half equivalent dose concentrations of APAP at a four-hour interval.

In another embodiment of the invention, a method is provided for treating pain and symptoms of respiratory illness in a subject in need of such treatment comprising administering a pharmaceutically effective amount of a pharmaceutical formulation comprising an immediate release component, an extended release component, APAP, and one or more optional decongestants, one or more optional antitussives, and one or more expectorants wherein, when said formulation is administered to a subject, said formulation provides eight-hour bioequivalence of APAP compared to two consecutive administrations of one-half equivalent dose concentrations of APAP at a four-hour interval.

In another embodiment of the invention, a process is provided for preparing a pharmaceutical formulation of claim 1, said the process comprising the steps of: (a) blending acetaminophen, at least one water soluble binder, a disintegrant, and one or more optional API(s) to form an immediate release component; (b) granulating and milling a composition comprising at least one controlled release matrix polymer to form an extended release component; and (c) blending the immediate release component, the extended release component, one or more optional binders, one or more optional glidants, and one or more optional lubricants to form the pharmaceutical formulation.

These and other embodiments of the invention will be become apparent in light of the detailed description below.

### BRIEF DESCRIPTION OF THE DRAWINGS

FIG. 1 is a flow diagram for the manufacturing process for propoxyphene napsylate APAP modified release.

FIG. 2 illustrates the particle size distribution for the immediate release (IR) portion of propoxyphene napsylate APAP modified release dosage form (lot K050568).

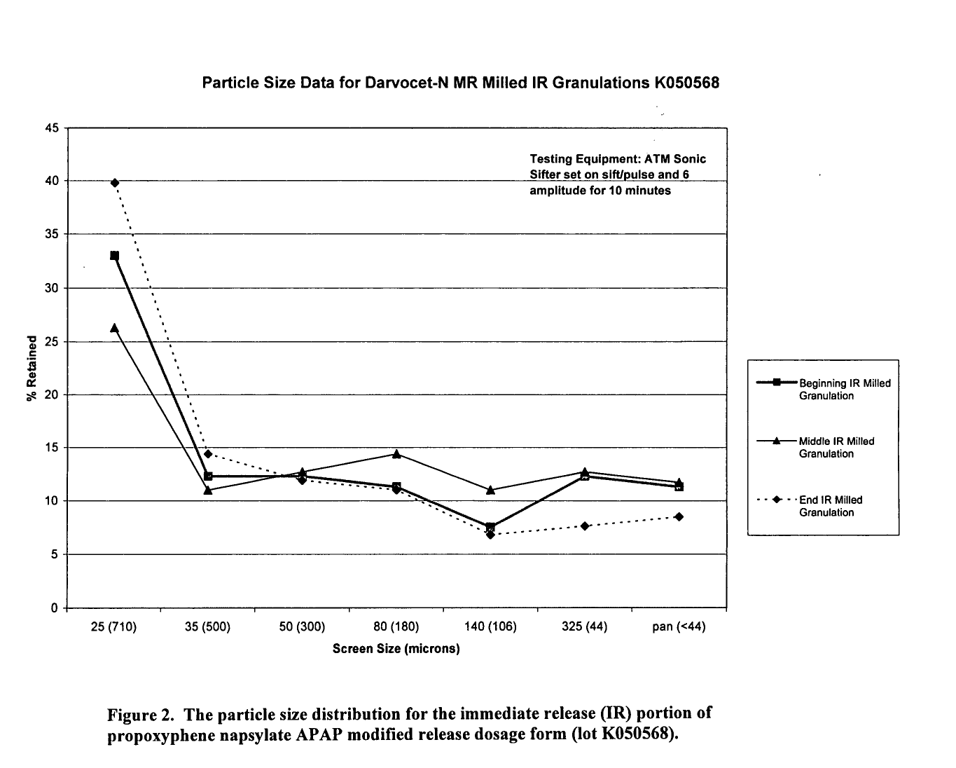

FIG. 3 illustrates the particle size distribution for the extended release (IR or XR) portion of propoxyphene napsylate APAP modified release dosage form (lot K050568).

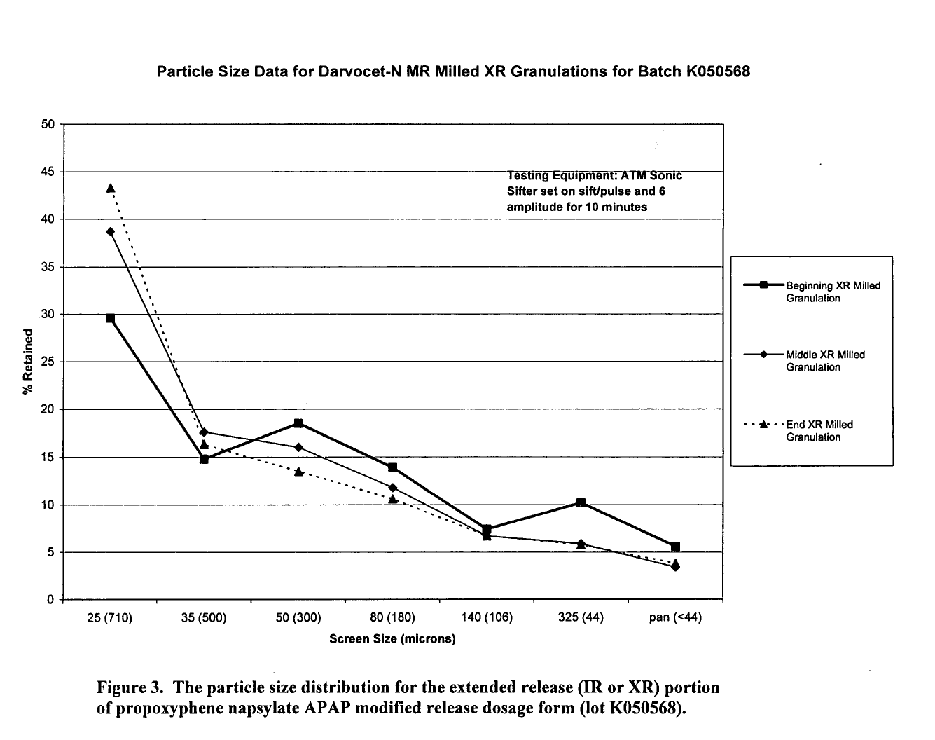

FIG. 4 illustrates the particle size distribution for the final blend of propoxyphene napsylate APAP modified release dosage form (lot K050568).

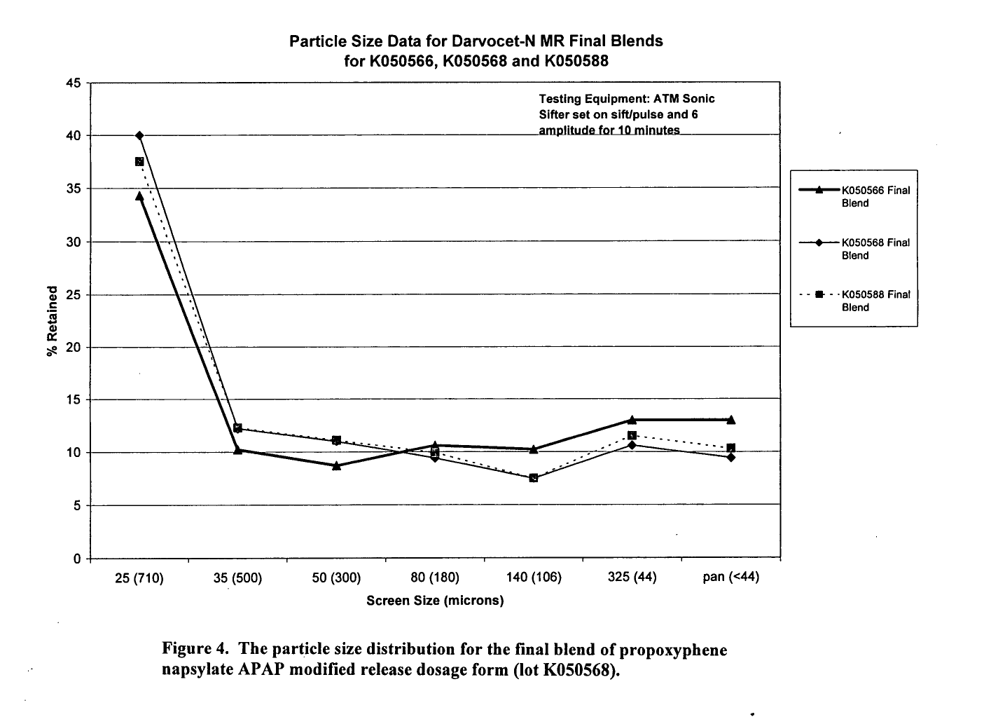

FIG. 5 illustrates the 99.9% confidence interval around the mean results for the concentrations at each sampling time for the single, fasted dose of propoxyphene in the propoxyphene napsylate APAP modified release dosage form for study PA425.

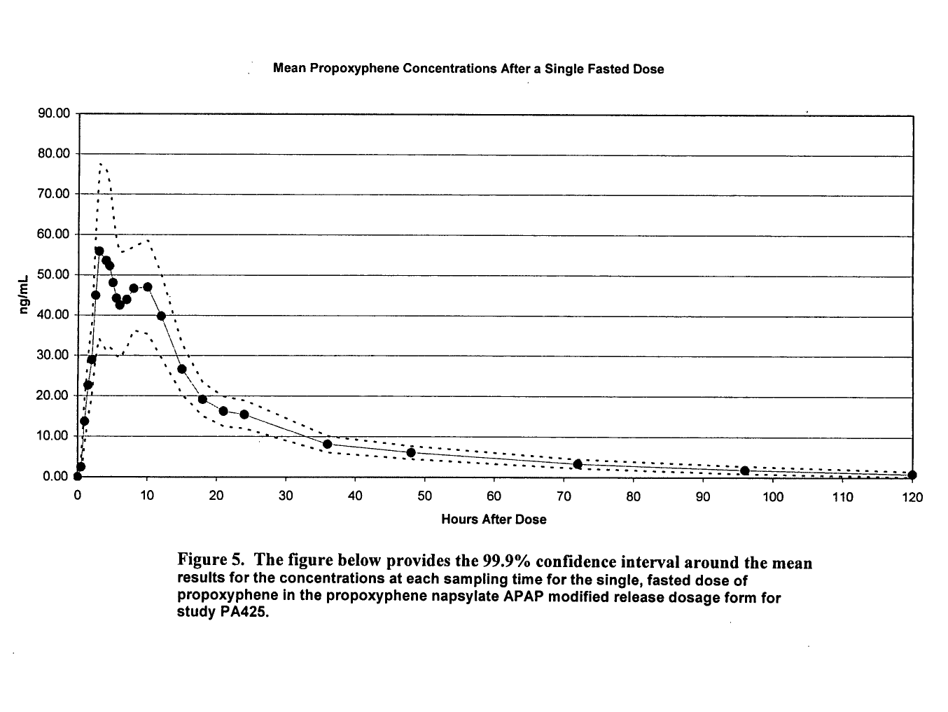

FIG. 6 illustrates the 99.9% confidence interval around the mean results for the concentrations at each sampling time for the single, fasted dose of acetaminophen in the propoxyphene napsylate APAP modified release dosage form for study PA425.

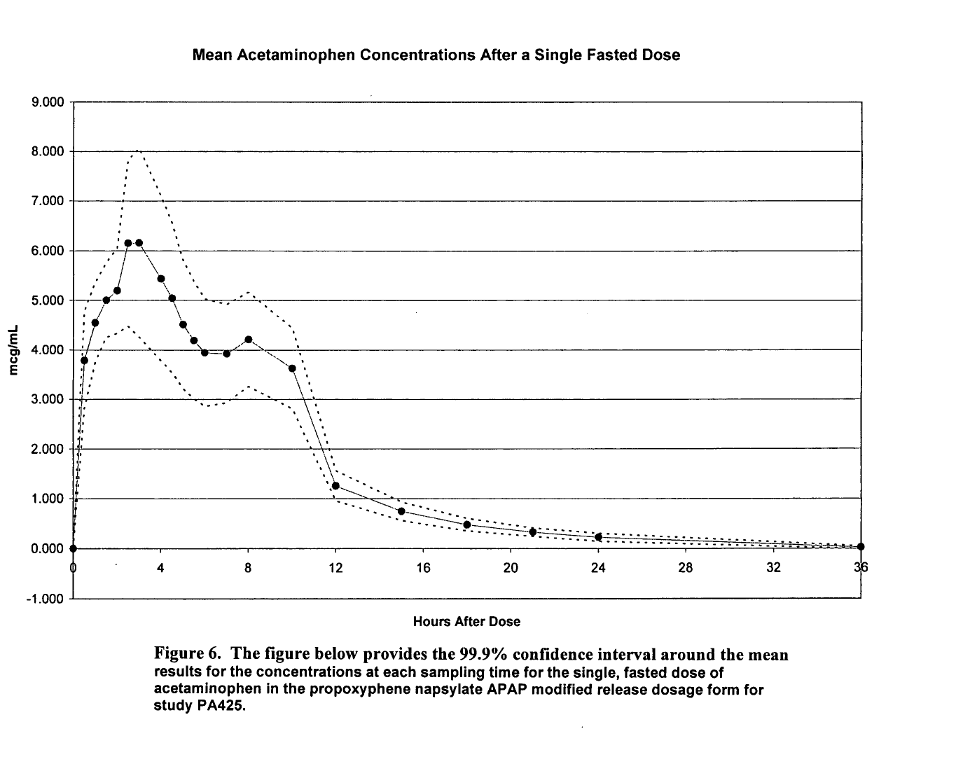

FIG. 7 illustrates the mean concentration of propoxyphene on Day 9 (0-8 h) for both Propoxyphene Napsylate APAP modified release tablet and Darvocet N 100 as described in Example 16.

FIG. 8 illustrates the mean concentration of acetaminophen (APAP) on Day 9 (0-8 h) for both Propoxyphene Napsylate APAP modified release tablet and Darvocet N 100 as described in Example 16.

### DETAILED DESCRIPTION OF THE INVENTION

Before the present invention is described in detail, it is to be understood that unless otherwise indicated this invention is not limited to specific pharmaceutical agents, excipients, polymers, salts, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include single and plural referents unless the context clearly dictates otherwise. Thus, for example, reference to “a carrier” may include one or more carriers; reference to “a pharmaceutical agent” may include one or more pharmaceutical agents, and so forth.

For clarity and convenience herein, the convention is utilized of designating the time of drug administration or initiation of dissolution testing as zero hours (t=0 hours) and times following administration in appropriate time units, e.g., t=30 minutes or t=2 hours, etc.

As used herein, the term “API” means a pharmaceutical active ingredient. Representative examples include acetaminophen, propoxyphene, hydrocodone, and oxycodone.

The term “AUCt” refers to the area under the concentration time curve to the least measurable concentration, estimated by the linear trapezoidal method.

The term “AUCinf” refers to AUCt extrapolated to infinity, calculated as AUCT +Ct/K, where Ct is the last measurable concentration and K is the apparent terminal rate of decay for the concentration-time profile.

The term “Cmax” refers to the plasma concentration of acetaminophen and/or an API(s) at Tmax expressed as ng/mL and ug/mL, respectively, produced by the oral ingestion of a composition of the invention. Unless specifically indicated, Cmax refers to the overall maximum observed concentration in the concentration-time profile.

The term “bioequivalence” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. See FDA: Guidance “Food Effect Bioavailability and Fed Bioequivalence Studies US Department of Health and Human Services Food and DrugAdministration, Center for Drug Evaluation and Research (CDER) December 2002 BP.

The term “dosage form” refers to a pharmaceutical composition comprising one or more active pharmaceutical ingredient (API), or a pharmaceutically acceptable acid salt, solvate, hydrate, coordination compound or combinations thereof, the composition optionally containing pharmacologically inactive ingredients, i.e., pharmaceutically acceptable carriers, fillers, excipients or combinations thereof such as polymers, suspending agents, surfactants, disintegrants, dissolution modulating components, binders, fillers, lubricants, glidants stabilizers, antioxidants, osmotic agents, colorants, plasticizers, coatings and the like, that are used to manufacture and deliver active pharmaceutical agents.

The term “extended-release” refers to the release of the API from the dosage form over a period of time compared to an immediate release dosage form. Generally the extended release occurs at such a rate that blood (e.g., plasma) concentrations in the patient administered the dosage form are maintained within the therapeutic range, that is, above the minimum effective analgesic concentration or “MEAC” but below toxic levels, over a period of time including, for example, 8, 12, or 24 hours.

The term “fasting conditions” means that following an overnight fast of at least 10 hours, subjects should be administered the drug product with 240 mL (8 fluid ounces) of water. No food should be allowed for at least 4 hours post-dose. Water can be allowed as desired except for one hour before and after drug administration. Subjects should receive standardized meals scheduled at the same time in each period of the study.

The term “immediate-release” refers to the substantially complete release of drug within a short time period following administration, e.g.., generally within a few minutes to about 1 hour.

The term “pharmaceutically acceptable salt” is meant those salts in which the counterion does not contribute significantly to the toxicity or pharmacological activity of the salt, and, as such, they are the pharmacological equivalent of the free form of the API(s). A multitude of non-toxic, pharmaceutically acceptable organic and inorganic acid and base addition salts are well known in the art.

The term “unit dosage form” refers to physically discrete units, such as capsules or tablets suitable as unitary dosages for human patients and other mammals, each unit containing a predetermined quantity of one or more active ingredient(s) calculated to produce the desired therapeutic effect, in association with at least one pharmaceutically acceptable carrier, diluent, excipient, or combination thereof.

The present invention provides modified release pharmaceutical dosage forms having eight-hour bioequivalence of APAP compared to two consecutive administrations of one-half equivalent dose concentrations of APAP administered at a four-hour interval. The compositions can be administered to a mammal, particularly a human patient, in a manner to provide effective concentrations of analgesic to provide an early onset of analgesic activity via the immediate release component of this invention sufficient to treat pain for about 8 hours.

In one embodiment of the invention, the modified release dosage form comprises a immediate release component and an extended release component, wherein the immediate release component and the extended release component collectively contain a therapeutically effective amount of APAP and optional API(s). The weight % (X) of APAP in the immediate release component generally ranges from about 90%, 95% and 100%. The remaining weight % balance of APAP (100-X) is present in the extended release component. Generally, the dosage forms release approximately 70%, 75%, 80%, 85%, 90%, or 95% of the APAP within the first hour after oral administration. The remaining APAP continues to be released for a period sufficient to provide 8 hours of therapeutic relief. If optional API(s) are included in the dosage form, the dosage form releases sufficient amounts of the API(s) to fall within the therapeutic window of effectiveness within the first hour of oral administration followed by steady release of the balance of the API sufficient to provide 8 hours of therapeutic relief. For propoxyphene napsylate or hydrochloride salts as the optional API, approximately 50% is released in the first hour. The therapeutic blood concentration and amount needed for release varies between API(s).

The modified release dosage forms may be prepared using standard techniques well known in the art and includes pharmaceutically acceptable carriers, fillers, excipients, or combinations thereof. Other ingredient commonly used in the pharmaceutical industry may also be included in the dosage forms such as preservatives, antioxidants, flavoring, coloring, and the like. As is well known to those skilled in the art, pharmaceutically acceptable carriers, fillers, excipients or combinations thereof are routinely incorporated into solid dosage forms, but in a great variety of combinations, each combination and ratios thereof and means by which to prepare the drug product are designed to provide desired characteristics to a respective dosage form. Generally, the immediate release component and extended release component are prepared separately then blended to form a final mixture. Optional binder, optional glidant, and optional lubricant, or combinations thereof, may be used. If desired, this final mixture may be loose filled into capsules or be compressed into tablet or caplet form. It will be understood by persons of ordinary skill in the art that a single drug formulation substance may be classified under different overlapping classifications such as a binder, a matrix polymer, a disintegrant and/or a wicking agent, depending on its particular use in a drug product. For clarification of the intended use of drug formulations substances, each classification term is defined below.

As defined herein, the “immediate release component” refers to a portion of the matrix composition that comprises acetaminophen, an optional binder, and a disintegrant. One or more additional API(s) may be included. The immediate release component may be prepared by dry blending acetaminophen and at least one optional binder, at least one disintegrant, and the optional API(s) to form an immediate release mixture. If desired, the immediate release mixture may be granulated and milled. Although wet or dry granulation techniques may be used, dry granulation is generally useful. The immediate release component has a mean particle size ranging from about 300 microns to about 1500 microns, usually about 300 to about 700 microns.

The term “binder” or “binders” means one or more substances that cause adhesion of powder particles in pharmaceutical tablet granulations. Exemplary binders include acacia, tragacanth, gelatin, starch, cellulose materials such as methyl cellulose and sodium carboxymethyl cellulose, hydroxypropylmethylcellulose (HPMC), hydropropylcellulose (HPC), hydroxycellulose, alginic acids and salts thereof, polyethylene glycol, guar gum, polysaccharide, bentonites, sugars, invert sugars, poloxamers (e.g., PLURONIC F68, PLURONIC F127), collagen, albumin, gelatin, starch, and pregelatinized starch, cellulosics in nonaqueous solvents, xanthum gum, combinations thereof and the like. Other binders include, for example, polypropylene glycol, polyoxyethylene-polypropylene copolymer, polyethylene ester, polyethylene sorbitan ester, polyethylene oxide, poly(vinylpyrrolidone), compressible sugar (e.g., NuTab), ethylcellulose, liquid glucose, povidone and pregelatinized starch combinations thereof and other materials known to one of ordinary skill in the art. In general, the amount of binder used in the composition is not more than 5% (w/w) of the IR component and not more than 10%(w/w) of the total dosage form.

The term “disintegrant(s)” generally refers to one or more substances that reacts with aqueous solutions including gastric juices to rapidly promote the disintegration of the immediate release portion of the tablet/capsule. Generally, disintegrants are used in solid pharmaceutical dosage forms to promote the disruption of the solid mass into smaller particles which are more readily dispersed or dissolved. Exemplary disintegrants include starches such as corn starch, potato starch, pre-gelatinized and modified starches thereof, sweeteners, clays, such as bentonite, microcrystalline cellulose (e.g., AVICEL), carboxymethylcellulose calcium, cellulose polyacrilin potassium (e.g., AMBERLITE), alginates, sodium starch glycolate, crospovidone, gums such as agar, guar, locust bean, karaya, pectin, tragacanth and other materials known to one of ordinary skill in the art. In general, the amount of disintegrant used ranges from about 14% to 23%, usually about 17% to 21% (w/w) by weight of the immediate release component.

As defined herein, the “extended release component” refers to a portion of the dosage form composition that comprises acetaminophen and at least one controlled release matrix polymer. Optional filler and optional wicking agents, or both, may be employed. One or more optional API(s) may be included if desired. The extended release component may be prepared by blending acetaminophen and the optional API(s) with at least one controlled release matrix polymer to form an extended release mixture. The extended release mixture is then granulated and blended to form the extended release component. The extended release component has a mean particle size ranging from about 300 microns to about 1500 microns, usually about 500 to about 800 microns.

The term “Controlled release matrix polymer” generally means one or more substances that forms matrix regions in the capsule/tablets in intimate contact with the API(s) that retard the release of the API(s) in a controlled manner. Matrix polymers include water-soluble hydrophilic polymer, water-insoluble hydrophilic polymer, water insoluble hydrophobic polymer or nonpolymer waxes. Examples of suitable water soluble polymers include polyvinylpyrrolidine, hydroxypropylcellulose (HPC), hydroxypropylmethyl cellulose (HPMC), hydroxyethylcellulose, hypromellose, methyl cellulose, vinyl acetate copolymers, polysaccharides (such as sodium alginate, xanthum gum, etc.), polyethylene oxide, high molecular weight polyethylene glycols (MW 1000 and above], acrylic and methacrylic acid copolymers, maleic anhydride/methyl vinyl ether copolymers and derivatives and mixtures thereof. When using hydroxypropylmethyl cellulose, a high viscosity hydroxypropylmethyl cellulose with a viscosity ranging from about 4,000 cps to about 100,000 cps (defined by a 2% w/v aqueous solution at 20° C.), usually with a viscosity of about 15,000 cps is useful and is commercially available as METHOCEL from The Dow Chemical Company, Midland, Mich., USA. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable waxes include fatty acids, fatty alcohols and glycerides, including stearic acid, cetyl alcohol, stearyl alcohol, bees wax, and carnuba wax. The amount of matrix polymer present in the extended release component generally ranges from about 20 to 60%, 30-50%, usually about 41% by weight relative to the weight of the dosage form.

Filler may be used in the immediate release component, extended release component or both. As defined herein, the term “filler” means one or more inert substances that dilutes the API(s) in the immediate release portion, provides additional bulk to the immediate release portion allowing the rapid intrusion of water and release of the API(s), and aids in providing the desired flow properties and compression characteristics. Such substances include, for example, powdered sugar, dibasic calcium phosphate, calcium sulfate, sodium chloride, kaolin, lactose, sucrose, mannitol, microcrystalline cellulose, powdered cellulose, precipitated calcium carbonate, sorbitol, and dry starch and other materials known to one of ordinary skill in the art. In general, the amount of filler used in the composition ranges from about 0% to 50% by weight relative to the weight of each of the immediate release component, the extended release component or both.

While wicking agents are generally considered in the art to be a subclass of disintegrants, wicking agents, as defined herein, the term “wicking agent” means one or more substances that is present in the extended release portion of the tablet and that encourages adequate wetting of the polymer system after the initial release ensuring matrix formation. Specifically, wicking agents function to rapidly hydrate the dosage form in vivo so that the immediate release component can be dispersed rapidly into gastric juices as well as to activate the extended release component by facilitating the hydration and swelling of the matrix polymer to form an activated modified release rate-controlling matrix. The activated matrix slowly erodes, allowing for passive diffusion of API into gastric juices a controlled manner. Examples of suitable wicking agents include, by way of example and without limitation, croscarmellose sodium and crospovidone. In general, the amount of wicking agent used in the composition ranges from about 0% to 40%, usually 0% to 25% (w/w) by weight relative to the weight of the extended release component. Some API(s) may function as a wicking agent, e.g., propoxyphene napsylate, and therefore, a separate wicking agent may not be necessary to include in the dosage form.

The immediate release component and extended release component prepared as described above are then blended to form a final mixture. Though optional, lubricants, binders and glidants, and any combinations thereof, may be included in the mixture. The final mixture may be loose filled, for example, into capsules or sachets. If desired, the final mixture may also be compressed, for example, into tablet or caplet forms.

In one embodiment, the modified release dosage forms comprise an immediate release component and an extended release component, wherein the immediate release component and the extended release component collectively contain a therapeutically effective amount of APAP and a therapeutically effective amount of one or more optional API(s) such as, for example, an opioid or opioid-like analgesic. The ratio of APAP to at least one API can vary, depending upon the desired therapeutic dose and potency of the API(s).

In certain embodiments, the opioid or opioid-like analgesic comprises alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, or salts of any of the foregoing.

In one aspect of this embodiment, the opioid analgesic is selected from the group consisting of propoxyphene (Napsylate or HCL), hydrocodone, morphine, hydromorphone, oxycodone, codeine, levorphanol, meperidine, methadone, or salts thereof, or mixtures thereof.

In addition to opiod or opioid-like analgesic drugs, other representative API(s) include non-steroidal anti-inflammatory drugs (NSAID), cyclooxygenase-II (COX-2) inhibitors, glycine receptor antagonists, antitussives, expectorants, decongestants, antihistamines or mixtures thereof.

Representative NSAID compounds include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, or salts of any of the foregoing.

Representative COX-2 inhibitors include, without limitation, celecoxib (SC-58635), DUP-697, flosulide (CGP-28238), meloxicam, 6-methoxy-2 naphthylacetic acid (6-MNA), MK-966, nimesulide, NS-398, SC-5766, SC-58215, T-614; or combinations thereof.

Representative glycine receptor antagonists and the use of such drugs in treating pain are known and are described, for instance, in U.S. Pat. No. 5,514,680 which is included by reference in its entirety.

Representative antihistamines and decongestants include pyrilamine, chlorpheniramine, cimetidine, tetrahydrozoline, loratadine, antazoline, chlorpheniramine maleate, clemastine fumarate, mequitazirie, alimemazine tartrate, cycloheptazine hydrochloride, bepotastine besilate and the like.

Representative expectorants include bromhexine hydrochloride, carbocysteine, ethylcysteine hydrochloride, methylcysteine hydrochloride and the like;

Representative antitussives include tipepidine hibenzoate, methylephedrine hydrochloride codeine phosphate, tranilast, dextromethorphan hydrobromide, dimemorfan phosphate, clobutinol hydrochloride, fominoben hydrochloride, benproperine phosphate, eprazinone hydrochloride, chlophedianol hydrochloride, ephedrine hydrochloride, noscapine, pentoxyverine citrate, oxeladine citrate, isoaminil citrate and the like.

In one aspect of this embodiment, the modified release dosage forms comprise an immediate release (IR) component and an extended release (ER) component, wherein the immediate release component and the extended release component collectively contain a therapeutically effective amount of APAP and a therapeutically effective amount of propoxyphene as the optional API. Examples of weight ratios [TRUE?] of propoxyphene in the IR and ER component include 50%IR:50%ER, 45%IR:55% ER, and 40%IR:60%ER. These ratios may be applied to other opioid or opioid-like compounds but are not considered to be limiting.

It has been discovered that a single administration of the dosage form of the present invention to healthy human subjects results in blood levels of APAP that is consistent with blood levels provided two consecutive admininstrations of one-half equivalent dose concentrations of a comparator product DARVOCET® N, a APAP/Propoxyphene napsylate formulation sold by Xanadyne Pharmaceuticals, Inc., over a four-hour interval. For a representative single dose of two tablets that includes includes 1300 mg APAP and 200 mg propoxyphene napsylate, the APAP/propoxyphene napsylate dosage forms produce patient plasma profiles with a 99.9% confidence interval characterized by a Cmax for propoxyphene ranging from about 56 to about 98 ng/mL/mg and an AUCinf for propoxyphene ranging from about 992 to about 1530 ng/mL/hr and a Cmax for acetaminophen ranging from about 5.89 to about 9.52 ug/mL/mg and an AUCinf for acetaminophen ranging from about 55.65 to about 73 ug/mL/hr after a single dose. Following administration to a patient, the single dose of APAP/proxyphene napsylate having a 99.9% confidence interval produces a Cmax for propoxyphene ranging from about 20.0 to about 40.0 ng/mL/mg and a Cmax for APAP ranging from about 4.0 to about 7.0 ug/mL/hour at t=2 hours; Cmax for propoxyphene ranging from about 31.0 to about 76.0 ng/mL/mg and a Cmax for APAP ranging from about 3.0 to about 8.0 ug/mL/hour at t=4 hours; Cmax for propoxyphene ranging from about 29.0 to about 56.0 ng/mL/mg and a Cmax for APAP ranging from about 2.0 to about 6.0 ug/mL/hour at t=6 hours; and Cmax for propoxyphene ranging from about 36.0 to about 58.0 ng/mL/mg and a Cmax for APAP ranging from about 3.0 to about 5.2 ug/mL/hour at t=8 hours.

The compositions of the invention can be administered orally in the form of tablets, caplets, or granulate loose filled, for example, into capsules or sachets. The tablets can be prepared by techniques known in the art and contain a therapeutically useful amount of the acetaminophen, one or more optional API(s) such as an opioid or opiod-like analgesic and such excipients, fillers, and the like as are necessary to form the tablet by such techniques.

In another embodiment of the invention, a method for preparing a modified release pharmaceutical dosage form having an 8-hour bioequivalence of APAP compared to two consecutive administrations of one-half equivalent doses of concentrations of APAP at a four-hour interval is provided. According to the method, an immediate release component and an extended release component are prepared separately, then blended with additional lubricant with or without additional components as described above to form a final mixture. If desired, this final mixture may then be compressed under pressure to form a modified release tablet or caplet.

Acetaminophen (APAP) is regarded as an API with poor compressibility and flow properties. It is desirable to improve the flow and/or compressibility, which is particularly important when manufacturing a compressed tablet or other compressed or compressable dosage forms. Therefore it is desirable to incorporate a binder or other compressible excipients in the immediate release portion containing the vast majority of the APAP and in the final blend containing the immediate release portion, extended release portion and other excipients to aid processing. Flow and compressibility can be attained by using a commercially available source of APAP such as Compap L by Mallinckrodt Corp.,Greenville, Ill. which has been spray dried in combination with compressible sugar and a water soluble binder such as povidone. The immediate release portion containing APAP can also be made flowable and compressible by incorporating a binder and granulating using with a wet or dry technique. The binder may take the form of traditional binders such as povidone, hydroxypropyl cellulose (HCP) or hypromellose, or it might utilize the compressibility of other excipients with dual functions including compressible disintegrants or fillers such as cross-linked povidone or microcrystalline cellulose. Compressibility and flow is not as important when manufacturing a capsule, sachet or other dosage form which can utilize loose powder or granules and, in these cases, the binder in the immediate release portion and final blend is optional. In these dosage forms, the immediate release portion and final blend are not necessarily compressed and poor flow properties can be overcome with the use of proper processing equipment.

The immediate release component may be prepared by blending APAP, an optional water soluble binder, a disintegrant, and one or more optional API(s) to form an IR mixture. If desired, the IR mixture may be granulated and milled to form the immediate release component. Wet or dry granulation methods can be used. Wet granulation methods include the use of a liquid whereas liquid is substantially excluded in dry granulation methods. The dry granulation method typically includes the aggregation of powder particles under high pressure by slugging (large tablet formed on a tablet press) or roller compaction (squeeze powder through rollers to form a sheet or ribbon). Wet granulation methods involve the massing of mix of dry powder particules using a granulating fluid to form a wet mass. Generally, the wet mass is forced through a sieve to produce granules which are then dried. Wet granulation methods may include, for example, the use of low shear granulators, high shear granulators, rotor granulation (Freund granulator), fluid bed granulators, spray driers, extrusion and spheronization. In both wet and dry granulation processes, the granules are subject to milling or screening to achieve a mean particle size ranging from about 300 microns to about 1500 microns, usually about 300 to about 700 microns.

The extended release component may be prepared by blending acetaminophen with at least one controlled release matrix polymer to form an extended release mixture. If desired, optional filler, optional wicking agent or both may be included in the extended release mixture. The extended release mixture is then granulated and milled to form the extended release component. The extended release component generally has a mean particle size ranging from about 300 microns to about 1500 microns, usually about 500 to about 800 microns.

The immediate release component and the extended release component are then blended to form a final mixture. If desired, a lubricant, binder, and glidant,optionally including other pharmaceutically acceptable carriers, diluents, excipients, flavors, antioxidants, coloring agents and the like or any combination of the foregoing may be included.. The final mixture may be loose filled, for example, into capsules or sachets or may be further compressed by pressure, for instance, into a tablet or caplet.

Optional lubricants may be incorporated into a composition for a variety of reasons. As defined herein, the term “lubricant” refers to one or more substances used in pharmaceutical compositions to reduce friction during compression and to prevent sticking of the various components comprising a dosage form on tabletting and encapsulation equipment. Such compounds include calcium stearate, magnesium stearate, glyceryl behenate, polyethylene glycol, sodium stearyl fumarate, magnesium stearate, mineral oil, stearic acid, talc, synthetic magnesium silicate, fine grain silicon oxide, vegetable oil, zinc stearate, glyceryl monostearate, and other materials known to one of ordinary skill in the art, and the like. In general, the amount of lubricant used in the composition ranges from 0 to 5% of the drug product.

Optional glidants may also be incorporated into the composition. As used herein, the term “glidant” means one or more agent that improves powder flowability during granulation, encapsulation and/or tabletting. Representative examples include, without limitation, colloidal silica, cornstarch, talc, calcium silicate, magnesium silicate, silicon hydrogel, silicon dioxide, talc and other materials known to one of ordinary skill in the art. In general, the amount of glidant used in the composition ranges from about 0% to 1.0% (w/w) of the dosage form.

In another embodiment of the invention, methods are provided for treating a subject (i.e.,, mammal, particularly humans) comprising administering to a subject in need of such treatment a therapeutically effective amount of the pharmaceutical formulation described above. The pharmaceutical formulations described above can be used, for example, to provide an effective concentration of acetaminophen and one or more optional API(s) (i.e., opioid or opioid-like API(s) for treatment of pain in a subject in need thereof. The compositions can be administered to a human patient in a manner to provide effective concentrations of analgesic to quickly combat existing pain and to provide a modified release to maintain levels of analgesic sufficient to alleviate pain or minimize the possibility of breakthrough pain for about 8 hours. Moreover, the pharmaceutical formulations of the invention may be useful in co-administration with other analgesics such as 24 hour opiate-based pharmaceutical formulation. For instance, the pharmaceutical formulations of the invention may be administered prior to, concurrently with, or subsequent to administration of the other analgesic. The pharmaceutical formulations of the invention may also be used for the treatment or “prophylaxis” of pain in mammals and particularly humans. As used herein, the term “treatment”, or a derivative thereof, contemplates partial or complete inhibition of pain when a composition of the present invention is administered prophylactically or following the onset of pain for which such pharmaceutical formulation the present invention is administered. For the purposes of the invention, “prophylaxis” refers to administration of the active ingredient(s) to a mammal to protect the mammal from pain.

When formulated to include at least one decongestants, antitussives, expectorants, or antihistamines, or any combination thereof, the pharmaceutical formulations of the invention can also be used to used to treat a disease state resulting of elevated histamine levels as well as dry cough and nasal congestion caused by the common cold, flu, or other respiratory illness.

The typical active daily dose of the pharmaceutical formulation of the invention will depend on various factors such as, for example, the individual requirement of each patient. An attending physician may adjust the dosage rate based on these and other criteria if he or she so desires. Generally, the modified release dosage forms are suitable for daily oral dosing (e.g., three times (tid) daily oral dosing) to human patients. Although individual oral dosage forms may encompass any suitable therapeutically effective amounts of APAP, i.e., amounts of 325, 500, or 650 mg APAP, typically administered in one single dose or equally divided doses, are useful. Any therapeutically suitable total daily dosage amounts may be encompassed such as 2 grams, 3 grams, or 4 grams. It should be appreciated that daily doses other than those described above may be administered to a subject, as appreciated by an attending physician.

The dosage forms of the invention are suitable for oral administration. Suitable solid dosage forms for oral administration include, for example, capsules (e.g., hard or soft gelatin capsules), tablets, caplets, sachets, microencapsulates, powders, and granules. In such solid dosage forms, the granules may be loose filled into capsules or sachets or further compressed under pressure into tablets or caplets. Solid dosage forms may also be prepared with coatings and shells, providing such coatings and/or shells do not substantially affect the release of the active ingredients.

The following examples are intended to illustrate the invention, and are not to be construed as limiting the scope of the invention.

### EXAMPLE 1

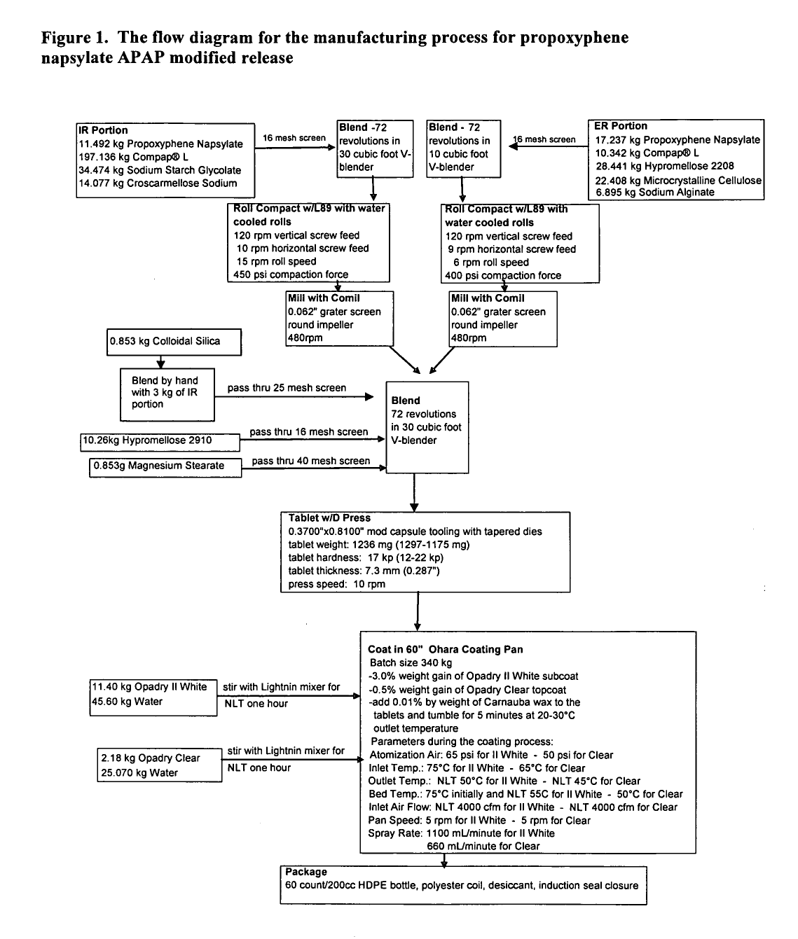

The following examples provide a manufacturing description for the preparation of propoxyphene napyslate acetaminophen (APAP) modified release dosage form. An overview of the manufacturing process is provided in Examples 1a-1f. The manufacture flow diagram is depicted for this manufacturing process in FIG. 1.

### EXAMPLE 1a

### Immediate Release Portion

1. Blend 11.492 kg propoxyphene napsylate, 197.136 kg Compap® L, 34.474 kg sodium starch glycolate and 14.077 kg of croscarmellose sodium for 72 revolutions in a 30 cubic foot blender.
2. Pass blended material through the roller compactor with a target roller speed of 15 rpm, a vertical screw speed target of 110-130 rpm (target 120 rpm), a horizontal screw speed of 0-30 rpm (target10 rpm) and a target roll cylinder pressure of 450 psi. The vertical and horizontal screw speeds may be adjusted within the specified ranges to obtain optimal ribbon thickness (0.031-0.041 inches) from the roller compactor.
3. Mill material with the Quadro™ Comil® using a 0.062″ grater screen and round impeller at 480 rpm.

### EXAMPLE 1b

### Modified Release Portion (ER)

4. Blend 17.237 kg propoxyphene napsylate, 10.342 kg Compap® L, 28.441 hypromellose 2208, 22.408 microcrystalline cellulose and 6.895 kg sodium alginate for 72 revolutions in a 10 cubic foot blender.
5. Pass blended material through the roller compactor with a target roller speed of 6 rpm, a vertical screw speed target of 110-130 rpm (target 120 rpm), a horizontal screw speed of 0-30 rpm (target 9 rpm) and a target roll cylinder pressure of 400 psi. The vertical and horizontal screw speeds may be adjusted within the specified ranges to obtain optimal ribbon thickness (0.050-0.060 inches) from the roller compactor.
6. Mill material with the Quadro™ Comil® using a 0.062″ grater screen and round impeller at 480 rpm.

### EXAMPLE 1c

### Final Blend

7. Remove approximately 3 kg of the immediate release portion and set aside.
8. Charge the remaining immediate and modified release portions into the 30 cubic foot blender.
9. Mix the 0.853 kg colloidal silicon dioxide and the 3 kg of immediate release portions by hand in a bag. Screen the blended material through a #25 mesh screen.
10. Screen 10.26 kg of hypromellose 2910 through a #16 mesh screen and 0.853 kg of magnesium stearate through a #40 mesh screen.
11. Charge all screened materials (steps 9 and 10) into the 30 cubic foot container with the immediate and modified blends.
12. Blend for 72 revolutions.

### EXAMPLE 1d

### Compression, Coating and Packaging

13. Compress the final blend using a D Tablet press using a 0.3700 inch×0.8100 inch modified capsule shaped tablet tooling at 10 rpm, the target parameters including, weight: 1236 mg, hardness: 17 kp and thickness: 7.3 mm (0.287″).
14. Coat the tablets with a subcoat of Opadry® II White (3.0% weight gain) and a topcoat of Opadry® Clear (0.5% weight gain) followed by carnauba wax.
15. Bulk imprinted tablets from each batch were packaged in double line LDPE bags with dessicant in a box for stability purposes.
16. Imprinted tablets from each batch were packaged 6 tablets per bottle in 30 cc HDPE bottles with dessicant polyester coil and induction sealed CRC closures for stability.
17. Imprinted tablets from each batch were packaged 60 tablets per bottle in 200 cc HDPE bottles with dessicant, polyester coil and induction sealed CRC closures for stability.
18. Imprinted tablets from each batch were packaged 500 tablets per bottle in 1250 cc HDPE bottles with dessicant, polyester coil, and an induction seal cap and placed on stability.
19. Package tablets in bottles of 60 count in 200 cc HDPE bottles with polyester coil and a 3 gram desiccant with a child-resistant cap and induction seal for clinical blinding use.

### EXAMPLE 1e

### In-Process Controls

In-process controls occur at several stages of the manufacturing process. The following is a list of in-process controls that are established and followed during the manufacture of propoxyphene napyslate APAP modified release tablets:
1. Blend sampling and testing was performed on the blend prior to compression. Two samples of 2.5 g to 4.0 g were sampled from each drum. The mean of the blend uniformity was within the range of 90.0 to 110.0% label claim with a Relative Standard Deviation of ≦5.0%.
2. Physical tablet testing was performed during compression. Appearance and the average weight variation of 10 units were performed every 15 minutes and individual weight variations of 20 units, hardness and thickness were performed every 30 minutes. Friability testing was performed at the beginning, middle and end of the run. Confirmation of appropriate parameters was performed prior to compression of the entire blend. Specifications are provided below:
Appearance:
   White, capsule shaped tablets with no deboss on either side.
      Weight Variation of 10 units:
      Not more than (NMT) ±5% of theoretical weight per unit
      Limits: Minimum 1.174 g Maximum 1.297 g
   1. Individual Weight Variation of 20 units:
      Average weight of 20 units
      NMT ±5% of theoretical weight per unit
      Limits: Minimum 1.174 g Maximum 1.297 g
   2. Individual weight variations of 20 units
      NMT 2 of 20 are outside ±5% of theoretical weight per unit
      None are outside ±10% of the average weight
         Hardness: 12.0 kp-22.0 kp Target: 17.0 kp
      Thickness: 7.0 mm-7.6 mm Target 7.3 mm
      Friability: Loss is NMT 1.0% after 100 revolutions

### EXAMPLE 1f

### Container/Closure System

a. Packaging and Labeling of Clinical Supplies

Propoxyphene napyslate APAP modified release tablets to be used for the clinical trial and for stability studies was packaged 60 count in 200 cc HDPE bottles with desiccant, coil and induction seal child-resistant. Table 1 provides a more detailed description of the container closure system.

TABLE 1
Container closure system for propoxyphene napyslate APAP modified
release tablets

| Component | Supplier | Specification | DMF# |
|---|---|---|---|
| 38/400 200 cc Wide-Mouth | Quality | 6133 | 5798 |
| Round, Recessed-Panel, | Container | | |
| Opaque-White HDPE Bottle | | | |
| 38/400 White Polypropylene | Owens | 5134 | 2229 |
| Clic-Loc III Ribbed Cap with | | | (10562 - |
| HS-130 Liner and Red | | | resin) |
| Highlighted Letters | | | (885 - resin) |
| | | | (1378 - liner) |
| Can 2IN1 3G Desiccant | Sud-Chemie | 8636 | 2880 |
| Polyester Coil 12 Gram/yd | Carolina | 8637 | 4164 |
| (29615 or 103128) | Cotton | | |

### EXAMPLE 2

The quantitative formula for the preparation of propoxyphene napsylate APAP modified release product is listed below in Table 2.

TABLE 2

| | Propoxyphene napyslate APAP | |
|---|---|---|
| | modified release 100/650 | |
| | Unit Dose | |
| Ingredient | (mg/tablet) | |
| 1.1 PROPOXYPHENE NAPSYLATE | 100.0 | |
| Compap ® L | 722.2 | |
| Sodium starch glycolate | 120.0 | |
| croscarmellose sodium | 49.0 | |
| hypromellose 2208 | 99.0 | |
| microcrystalline cellulose | 78.0 | |
| Sodium alginate | 24.0 | |
| hypromellose, Type 2910 | 37.3 | |
| colloidal silicon dioxide | 3.1 | |
| Magnesium stearate | 3.1 | |
| Total | 1235.7 | mg |
| Coating | | |
| Opadry ® II White Y-22-7719 | 37.1 | |
| Opadry ® Clear YS-1-7006 | 6.2 | |
| Purified water | Removed during process | |
| Carnauba Wax | trace^{a} | |
| Total | 1279 | mg |

^{a}Carnauba wax is 0.01% w/w of the core tablet weight.

### EXAMPLE 3

The quantitative batch formula for the preparation of a 355 kg clinical batch for propoxyphene napyslate APAP modified release tablets is listed in Table 3 below.

TABLE 3

| | Propoxyphene napyslate | |
|---|---|---|
| | APAP modified release | |
| | tablets 100/650 | |
| | 355 kg Batch | |
| Ingredient | (g/batch) | |
| 1.2 PROPOXYPHENE | 28,729 | |
| NAPSYLATE | | |
| Compap ® L | 207,478 | |
| Sodium starch glycolate | 34,474 | |
| croscarmellose sodium | 14,077 | |
| Hypromellose 2208 | 28,441 | |
| microcrystalline cellulose | 22,408 | |
| Sodium alginate | 6,895 | |
| hypromellose, Type 2910 | 10,716 | |
| colloidal silicon dioxide | 891 | |
| Magnesium stearate | 891 | |
| Total | 355,000 | g |
| Coating | | |
| Opadry ® II White Y-22-7719 | 11,400^{a} | |
| Opadry ® Clear YS-1-7006 | 2,180^{a} | |
| Purified water | Removed during process | |
| Carnauba Wax | 34^{a} | |
| Opacode Orange Monogramming Ink | Trace | |
| Total | 368,614 | g |

^{a}g/batch is calculated based on a pan load containing 340 kg of propoxyphene napyslate APAP modified release tablets. 6 kg excess prepared for each Opadry ® II White and Opadry ® Clear to account for losses during transfer.

### EXAMPLE 4

The batch distribution for propoxyphene napyslate APAP modified release between IR, ER and extragranular portions are provided for the quantitative batch formula for a 375 kg clinical batch as listed in Table 4 below.

TABLE 4

| | | | | | Amount per | |
|---|---|---|---|---|---|---|
| Compartment | Material | mg/tablet | | w/w % | 375 kg batch | |
| Immediate Release | Compap L (90% acetaminophen) | 686.2 | | 55.5 | 208.24 | kg |
| Granulation | Propoxyphene Napsylate | 40.0 | | 3.2 | 12.14 | kg |
| | Croscarmellose Sodium | 49.0 | | 4.0 | 14.87 | kg |
| | Sodium Starch Glycolate | 120.0 | | 9.7 | 36.42 | kg |
| | | 895.2 | | 72.4% | 271.67 | |
| Extended Release | Compap L (90% acetaminophen) | 36.0 | | 2.9 | 10.92 | kg |
| Granulation | Propoxyphene Napsylate | 60.0 | | 4.9 | 18.21 | kg |
| | Microcrystalline Cellulose (Avicel | 78.0 | | 6.3 | 23.67 | kg |
| | PH 200) | | | | | |
| | Sodium Alginate | 24.0 | | 1.9 | 7.28 | kg |
| | HPMC K15M | 99.0 | | 8.0 | 30.04 | kg |
| | | 297.0 | | 24.0% | 90.84 | |
| Extragranular | HPMC E3 | 37.3 | | 3.0 | 11.32 | kg |
| | Cab-o-sil | 3.1 | | 0.3 | 0.941 | kg |
| | Magnesium Stearate | 3.1 | | 0.3 | 0.941 | kg |
| | Total | 1235.7 | mg | 100.0% | 374.99 | |
| Film Coat | Opadry II White, Grade Y-22-7719 | 37.1 | | 3.0% weight | 11.28 | kg |
| | | | | gain | | |
| | Opadry Clear, Grade YS-1-7006 | 6.2 | | 0.5% weight | 1.87 | kg |
| | | | | gain | | |
| | carnauba wax | trace | | trace | 34 | g |
| Inprinting | Opacode Orange Monogramming Ink | trace | | trace | trace | |
| | (TS31608) | | | | | |
| | Total | 1278.9 | | 103.50% | 388.17 | kg |

### EXAMPLE 5

The quantitative formula for the preparation of APAP modified release product is provided in Table 5 below. The method of preparation would be similar to Example 1a-1f. The manufacture process flow diagram would be similar to FIG. 1 with the exception of propoxyphene napsylate present in the formulation.

TABLE 5

| | Propoxyphene napyslate APAP | |
|---|---|---|
| | modified release 100/650 | |
| | Unit Dose | |
| Ingredient | (mg/tablet) | |
| Compap ® L | 722.2 | |
| Sodium starch glycolate | 120.0 | |
| croscarmellose sodium | 49.0 | |
| hypromellose 2208 | 99.0 | |
| microcrystalline cellulose | 78.0 | |
| Sodium alginate | 24.0 | |
| hypromellose, Type 2910 | 37.3 | |
| colloidal silicon dioxide | 3.1 | |
| magnesium stearate | 3.1 | |
| Total | 1135.7 | mg |
| Coating | | |
| Opadry ® II White Y-22-7719 | 37.1 | |
| Opadry ® Clear YS-1-7006 | 6.2 | |
| Purified water | Removed during process | |
| Carnauba Wax | trace^{a} | |
| Total | 1179 | mg |

^{a}Carnauba wax is 0.01% w/w of the core tablet weight.

### EXAMPLE 6

Table 6 provides the particle size distribution by sieve analysis for the immediate release (IR) portion of propoxyphene napsylate APAP modified release dosage form (lot K050568).

TABLE 6
Lot K050568

| | | | | AAI |
|---|---|---|---|---|
| | | | | Prototype |
| Mesh | IR Begin | IR Middle | IR End | 05035 |
| 25.0 | 34.6 | 26.3 | 39.8 | 33.0 |
| 35.0 | 15.4 | 11.0 | 14.4 | 13.2 |
| 50.0 | 14.4 | 12.7 | 11.9 | 11.3 |
| 80.0 | 11.5 | 14.4 | 11.0 | 9.4 |
| 140.0 | 7.7 | 11.0 | 6.8 | 5.7 |
| 325.0 | 8.7 | 12.7 | 7.6 | 11.3 |
| pan | 7.7 | 11.7 | 8.5 | 16.0 |

### EXAMPLE 7

Table 7 provides the particle size distribution by sieve analysis for the extended release (ER or XR) portion of propoxyphene napsylate APAP modified release dosage form (lot K050568).

TABLE 7

| | | | | AAI |
|---|---|---|---|---|
| | | | | Prototype |
| Mesh | XR Begin | XR Middle | XR End | 05035 |
| 25.0 | 38.3 | 38.7 | 43.3 | 35.9 |
| 35.0 | 15.9 | 17.6 | 16.3 | 13.6 |
| 50.0 | 13.1 | 16.0 | 13.5 | 15.5 |
| 80.0 | 11.2 | 11.8 | 10.6 | 10.6 |
| 140.0 | 7.4 | 6.7 | 6.7 | 6.8 |
| 325.0 | 8.4 | 5.9 | 5.8 | 10.6 |
| pan | 5.6 | 3.4 | 3.8 | 6.8 |

### EXAMPLE 8

Table 8 provides the particle size distribution by sieve analysis, bulk and tap density and percent compressibility for the final blend of propoxyphene napsylate APAP modified release dosage form (lot K050568).

TABLE 8
Final Blend Particle Size Data

| | % retained | | |
|---|---|---|---|
| Mesh | lot K050566 | lot K05068 | Lot K050588 |
| 25 | 34.3 | 40 | 37.5 |
| 35 | 10.2 | 12.2 | 12.3 |
| 50 | 8.7 | 11 | 11.1 |
| 80 | 10.6 | 9.4 | 9.9 |
| 140 | 10.2 | 7.5 | 7.5 |
| 325 | 13 | 10.6 | 11.5 |
| pan | 13 | 9.4 | 10.3 |
| Bulk (g/mL) | 0.63 | 0.63 | 0.64 |
| Tap (g/mL) | 0.79 | 0.82 | 0.82 |
| Compressibility | 20% | 24% | 22% |

### EXAMPLE 9

Table 9 below provides the shore hardness and tablet thickness in-process results for propoxyphene napsylate APAP modified release dosage form, batches K050566, K050568 and K050588.

TABLE 9

| Lot | Sample | Shore A Hardness | Thickness (inches) |
|---|---|---|---|
| K050568 | Beg IR | 99 | 0.038 |
| | Mid IR | 99 | 0.034 |
| | End IR | 99 | 0.034 |
| | Beg XR | 100 | 0.050 |
| | Mid XR | 100 | 0.056 |
| | End XR | 100 | 0.055 |
| K050588 | Beg IR | 99 | 0.036 |
| | Mid IR | 99 | 0.039 |
| | End IR | 99 | 0.038 |
| | Beg XR | 100 | 0.054 |
| | Mid XR | 99 | 0.055 |
| | End XR | 99 | 0.054 |
| K050566 | Beg IR | 100 | 0.034 |
| | Mid IR | 100 | 0.035 |
| | End IR | 99 | 0.037 |
| | Beg XR | 99 | 0.052 |
| | Mid XR | 99 | 0.054 |
| | End XR | 100 | 0.049 |

### EXAMPLE 10

Table 10 below provides the 99.9% confidence interval around the mean results for the concentrations at each sampling time for the single, fasted dose of propoxyphene in the propoxyphene napsylate APAP modified release dosage form for study PA425.

TABLE 10

| Hour | 99.9% CI_Low | 99.9% CI_High |
|---|---|---|
| 0 | 0.00 | 0.00 |
| 0.5 | 0.05 | 4.77 |
| 1 | 8.13 | 19.25 |
| 1.5 | 14.52 | 30.66 |
| 2 | 19.50 | 38.32 |
| 2.5 | 29.04 | 60.78 |
| 3 | 34.16 | 77.54 |
| 4 | 31.29 | 75.79 |
| 4.5 | 32.09 | 72.37 |
| 5 | 31.28 | 64.82 |
| 5.5 | 29.99 | 58.35 |
| 6 | 29.32 | 55.60 |
| 7 | 31.71 | 55.91 |
| 8 | 36.17 | 57.01 |
| 10 | 35.26 | 58.60 |
| 12 | 29.45 | 50.11 |
| 15 | 20.45 | 32.83 |
| 18 | 15.01 | 23.21 |
| 21 | 12.52 | 19.86 |
| 24 | 11.95 | 18.85 |
| 36 | 6.06 | 10.12 |
| 48 | 4.44 | 7.68 |
| 72 | 2.23 | 4.45 |
| 96 | 0.92 | 2.78 |
| 120 | 0.21 | 1.51 |

### EXAMPLE 11

Table 11 below provides the 99.9% confidence interval around the mean results for AUCT, AUCINF and CMAX for the single, fasted dose of propoxyphene in the propoxyphene napsylate APAP modified release dosage form for study PA425.

TABLE 11

| Parameter | Mean | 99.9% CI_Low | 99.9% CI_High |
|---|---|---|---|
| AUCT | 1181.4 | 937.80 | 1425.00 |
| AUCINF | 1260.5 | 991.53 | 1529.47 |
| CMAX | 77.51 | 56.10 | 98.92 |

### EXAMPLE 12

Table 12 below provides the 99.9% confidence interval around the mean results for the concentrations at each sampling time for the single, fasted dose of acetaminophen in the propoxyphene napsylate APAP modified release dosage form for study PA425.

TABLE 12

| Hour | 99.9% CI_Low | 99.9% CI_High |
|---|---|---|
| 0 | 0.00 | 0.00 |
| 0.5 | 2.79 | 4.78 |
| 1 | 3.74 | 5.36 |
| 1.5 | 4.25 | 5.76 |
| 2 | 4.33 | 6.05 |
| 2.5 | 4.48 | 7.82 |
| 3 | 4.26 | 8.06 |
| 4 | 3.78 | 7.09 |
| 4.5 | 3.54 | 6.55 |
| 5 | 3.22 | 5.81 |
| 5.5 | 3.01 | 5.37 |
| 6 | 2.85 | 5.03 |
| 7 | 2.92 | 4.92 |
| 8 | 3.26 | 5.16 |
| 10 | 2.81 | 4.44 |
| 12 | 0.96 | 1.56 |
| 15 | 0.56 | 0.93 |
| 18 | 0.36 | 0.60 |
| 21 | 0.24 | 0.42 |
| 24 | 0.14 | 0.31 |
| 36 | 0.00 | 0.05 |

### EXAMPLE 13

Table 13 below provides the 99.9% confidence interval around the mean results for AUCT, AUC_{INF} and C_{MAX} for the single, fasted dose of acetaminophen (APAP) in the propoxyphene napsylate APAP modified release dosage form for study PA425.

TABLE 13

| Parameter | Mean | 99.9% CI_Low | 99.9% CI High |
|---|---|---|---|
| AUCT | 64.484 | 55.04 | 73.93 |
| AUCINF | 64.190 | 55.65 | 72.73 |
| CMAX | 7.706 | 5.89 | 9.52 |

### EXAMPLE 14

The following examples provide a manufacturing description for the preparation for a capsule of propoxyphene napyslate acetaminophen (APAP) modified release dosage form. An overview of the manufacturing process is provided in Examples 14a-1f. The manufacture flow diagram is depicted for this manufacturing process in FIG. 1 with the exception of compression.

### EXAMPLE 14a

### Immediate Release Portion

1. Blend 11.492 kg propoxyphene napsylate, 197.136 kg Compap® L, 34.474 kg sodium starch glycolate and 14.077 kg of croscarmellose sodium for 72 revolutions in a 30 cubic foot blender.
2. Pass blended material through the roller compactor with a target roller speed of 15 rpm, a vertical screw speed target of 110-130 rpm (target 120 rpm), a horizontal screw speed of 0-30 rpm (target 10 rpm) and a target roll cylinder pressure of 450 psi. The vertical and horizontal screw speeds may be adjusted within the specified ranges to obtain optimal ribbon thickness (0.031-0.041 inches) from the roller compactor.
3. Mill material with the Quadro™ Comil® using a 0.062″ grater screen and round impeller at 480 rpm.

### EXAMPLE 14b

### Modified Release Portion (ER)

4. Blend 17.237 kg propox phene napsylate, 10.342 kg Compap® L, 28.441 hypromellose 2208, 22.408 microcrystalline cellulose and 6.895 kg sodium alginate for 72 revolutions in a 10 cubic foot blender.
5. Pass blended material through the roller compactor with a target roller speed of 6 rpm, a vertical screw speed target of 110-130 rpm (target 120 rpm), a horizontal screw speed of 0-30 rpm (target 9 rpm) and a target roll cylinder pressure of 400 psi. The vertical and horizontal screw speeds may be adjusted within the specified ranges to obtain optimal ribbon thickness (0.050-0.060 inches) from the roller compactor.
6. Mill material with the Quadro™ Comil® using a 0.062″ grater screen and round impeller at 480 rpm.

### EXAMPLE 14c

### Final Blend

7. Remove approximately 3 kg of the immediate release portion and set aside.
8. Charge the remaining immediate and modified release portions into the 30 cubic foot blender.
9. Mix the 0.853 kg colloidal silicon dioxide and the 3 kg of immediate release portions by hand in a bag. Screen the blended material through a #25 mesh screen.
10. Screen 10.26 kg of hypromellose 2910 through a #16 mesh screen and 0.853 kg of magnesium stearate through a #40 mesh screen.
11. Charge all screened materials (steps 9 and 10) into the 30 cubic foot container with the immediate and modified blends.
12. Blend for 72 revolutions.

### EXAMPLE 14d

### Fill Final Blend into a Capsule and Package

13. Fill the blend into an appropriate size capsule utilizing a manual or automated encapsulation equipment.
14. Packaged 6 capsules per bottle in 30 cc HDPE bottles and induction sealed CRC closures for stability.
15. Packaged 60 capsules per bottle in 200 cc HDPE bottles and induction sealed CRC closures for stability.
16. Packaged 500 capsules per bottle in 1250 cc HDPE bottles and an induction seal cap and placed on stability.
17. Package capsules in bottles of 60 count in 200 cc HDPE bottles with polyester coil and with a child-resistant cap and induction seal for clinical blinding use.

### EXAMPLE 15

This example provides a summary of the Clinical Study Report for an open-label, randomized, single dose, three-way cross-over, fasted comparative bioavailability study in healthy volunteers with an immediate and modified release propoxyphene/acetaminophen (APAP) formulation.
Title: An Open-Label, Randomized, Single Dose, Three-way Cross-over Fasted Comparative Bioavailability Study in Healthy Volunteers with an Immediate and Modified Release Propoxyphene/Acetaminophen Formulation.
EudraCT number 2005-004593-25
AAI Deutschland study code: PA425
Sponsor's study code XP20B-101
Phase: Phase I
Name of the investigational products: Test drug: propoxyphene napsylate acetaminophene (APAP) Modified Tablets Reference drug: Darvocet-N 100® Tablets
GCP statement: The study described within this report was performed in compliance with Good Clinical Practices

### The rights to the information in this document become the property of the Sponsor only upon completion of contractual obligations by AAI Deutschland GmbH & Co KG and the Sponsor.

### 2 Synopsis

### Objectives:

To assess the single-dose relative bioavailability of propoxyphene and acetaminophen (APAP) between the test drug (Propoxyphene Napsylate APAP Modified Release tablets or ER) and the reference drug (Darvocet N-100 Immediate Release tablets) evaluated under fasted conditions.

### Number of Subjects (Planned and Analyzed):

| | |
|---|---|
| Number of subjects planned: | 54 subjects |
| Number of subjects included: | 54 subjects |
| | 25 females |
| | 29 males |
| Number of subjects analyzed: | 50 subjects |
| pharmacokinetic data: | (4 subjects discontinued the study early) |
| | 24 females |
| | 26 males |

### Diagnosis and Main Criteria for Inclusion:

Healthy light smoking (≦10 cigarettes/day) and non-smoking, male and female volunteers of Caucasian race between 18 and 50 years of age, within ±15% of normal body weight for height, frame and gender.

### Test Drug, Dose and Mode of Administration, Batch Number:

Propoxyphene napsylate APAP modified release (ER) tablet, 100 mg propoxyphene napsylate/650 mg acetaminophen, batch number of manufacturer: K05068E, administered as a single two-tablet dose in fasted (treatment B) conditions

### Reference Drug, Dose and Mode of Administration, Batch Number:

Darvocet-N 100®, immediate release (IR) tablet, 100 mg propoxyphene napsylate/650 mg acetaminophen, batch number of manufacturer: A11788, administered as one-tablet doses at 0 and 4 hours in the morning under fasted (treatment C) conditions

### Criteria for Evaluation:

Efficacy: Not applicable

Pharmacodynamics: Not applicable

Pharmacokinetics: Pharmacokinetic parameters were calculated for acetaminophen, propoyphene and norpropoxyphene in plasma. Blood samples per period were collected at the following times: 0 (pre-dose) and 0.5, 1, 1.5, 2, 2.5, 3, 4, 4.5, 5, 5.5, 6, 7, 8, 10, 12, 15, 18, 21, 24, 36, 48, 72, 96 and 120 hours post-dose.

Primary variables: Area under the curve (AUC_{(0-∞)}, AUC₍₀₋ₜ₎), maximum concentration (Cₘₐₓ),

Secondary variables: time of maximum (tₘₐₓ), terminal half-life (t½), and terminal rate constant (Ke)

### Pharmacokinetic Results:

Relative bioavailability of propoxyphene and acetaminophen was assessed for a new modified release preparation (treatment B) in comparison to the immediate release tablet (treatment C, reference) in fasted subjects (n=50).

Results of the Modified Release (Fasting) vs. Immediate Release Tablets (Fasting)

### Ratios of Least Squares Means (90% Confidence Intervals)

### Treatment Arms B and C Included in the ANOVA

| Parameter | Acetaminophen | Propoxyphene |
|---|---|---|
| AUC inf | 96.0% (92.8–99.3%) | 113.3% (107.9–119.0%) |
| AUC 0–t | 95.4% (92.2–98.7%) | 114.0% (108.3–112.0%) |
| Cmax | 67.5% (61.4–74.1%) | 71.3% (64.2–79.1%) |

The extent of acetaminophen and propoxyphene absorption (AUC(0-inf) or AUC(0-t)) exhibited by the ER formulation of propoxyphene napsylate APAP is equivalent (ratio, and 90% confidenence interval) to the IR Darvocet N-100 reference formulation under fasting conditions. The lower Cmax for acetaminophen and propoxyphene exhibited by the Propoxyphene Napsylate APAP modified release dosage form (ER) relative to the IR Darvocet N-100 reference formulation is as expected when comparing a modified (extended) release to an immediate release formulation of the same product. Tmax of acetaminophen and propoxyphene produced by the ER product occurs much later relative to the IR product. Due to the plateau-like shape of the plasma concentration profile curves produced by the ER product neither tmax nor Cmax are suited for a detailed statistical comparison.

### Conclusion

Fifty-four (54) subjects were treated with study medication according to protocol. Two subjects withdrew from the trial after the second period and two subjects were withdrawn from the study at the time of check-in period 2 due to positive urine drug or an alcohol breath test. The remaining 50 subjects were dosed and completed the trial as planned. Full data sets are available for 50 subjects. The study was performed according to the study protocol in all relevant parts.

The relative bioavailability of propoxyphene and acetaminophen was assessed for a new modified release tablet preparation (treatment B) in comparison to an immediate release tablet (treatment C, reference) formulation in fasted subjects (n=50). Both treatments are equivalent with respect to their absorption (AUC_{(0-∞)} or AUC₍₀₋ₜ₎) for the two active drug ingredients (i.e. acetaminophen and propoxyphene). The lower Cmax for both active ingredients exhibited by the modified release tablet formulation of propoxyphene napsylate APAP relative to the immediate release reference tablet Darvocet N-100 formulation is as expected.

### 2.1 Rationale

There were no in vitro tests that were conclusive in assessing the bioavailability of propoxyphene/acetaminophen combination preparations. Therefore, a pharmacokinetic comparison study in human subjects had to be performed. A new modified form of Darvocet-N that contains 100 mg of propoxyphene napsylate (40 mg in immediate-release form, 60% as extended-release) and 650 mg of acetaminophen (formulated for approximately 50% to release immediately, 50% as extended release) was developed. The total daily dose of both the modified and currently marketed Darvocet-N 100® product was expected to be comparable. The modified release tablet, with its extended-release propoxyphene. napsylate and acetaminophen components, was designed to be dosed as two tablets every 8 hours, instead of one tablet every 4 hours as is the currently marketed product. The goal is to provide more convenient dosing (i.e., dosed less frequently) which produces less peak to trough plasma concentration fluctuation over the dosing interval while providing comparable total exposure and less or comparable peak exposure to the active drug ingredients (and metabolites).

Combinations of an opioid for weak and moderate pain and a non opioid analgesic are recommended by WHO for the treatment of moderate to severe pain stage 2. They recognize the use of these combinations for treating pain in subjects who do not require a strong opioid, but are not free of pain after treatment with NSAIDs or non-opioid analgesics alone. Combination therapy is a generally recognized medical treatment modality. By combining two different analgesics into one preparation, one expects an addition of the analgesic efficacy at the lowest possible dose of the single active agents. Combinations of a mild opioid and NSAIDs or acetaminophen are well accepted treatment modality.

The present study was designed as an open-label, randomized, single dose, food effect and comparative bioavailability evaluation of two different propoxyphene napsylate/acetaminophen combination tablets in 54 healthy volunteers. In addition, comparative safety and tolerance information were obtained for the propoxyphene napsylate APAP modified release formulation utilized in this study.

### 3 Study Objectives

The primary objectives of this study were to evaluate the comparative bioavailability of the modified release formulation to that of the marketed immediate release tablets under fasted conditions and to evaluate the effect of food on the bioavailability of propoxyphene and acetaminophen from the investigational propoxyphene napsylate modified release tablets.

### Primary Variables to be Studied in Detail Were:

Area under the curve (AUC_{(0-∞)}, AUC₍₀₋ₜ₎) and maximum concentration (Cₘₐₓ)

### Secondary Variables to be Studied Were:

Time of maximum (tₘₐₓ), terminal half-life (t_{1/2}), terminal rate constant (Ke)

### 4 Investigational Plan

### 4.1 Overall Study Design and Plan

Study Design and Schedule of Assessments

Treatment B: Propoxyphene Napsylate APAP Modified Release tablet (fasted dose) (n=54)

Treatment C: Darvocet-N 100®, Immediate Release marketed tablet (fasted dose) (n=54)

### 4.1.1.1 Dietary Regimen

### Fasted Treatment:

The subjects fasted from at least 10 hours before until 2 hours after drug administration in each study period (period 1, 2 and 3). A light, low-fat breakfast was given at 2 hours and then the next meal not until 6 hours after drug administration. They were not allowed to lie down for 4 hours after dosing. The drug product was administered with 240 mL (8 fl oz) of water. Water intake was ad lib except from 1 hour pre-dose to 1 hour post-dose. Six (6) hours after drug administration, a standardized lunch was served.

### Post-Study Testing

The post-study examination verified that all values tested pre-study had remained within a clinically acceptable range. The tests were performed after Period 3. Unacceptable values were followed up until they returned to the reference range or there was an adequate explanation, which was not related to the trial.

### Inclusion Criteria

Healthy light smoking (≦10 cigarettes/day) and non-smoking, male and female volunteers of Caucasian race between 18 and 50 years of age were recruited from the local population and fifty four (54) eligible subjects were included in the study after having given voluntary written informed consent before the first invasive pre-study examination procedure.

### Exclusion Criteria

History of:
drug hypersensitivity to propoxyphene, codeine, acetaminophen or similar NSAID compounds
seizure disorder
severe digestive disorder
surgery of the digestive tract (except for appendectomy)
liver function disturbances
renal disorders (albuminuria, chronic infections)
respiratory or cardiovascular disorders
diabetes mellitus
hyperthyroidism or other endocrine disorders
malignancy
recent history of mental illness
history of substance abuse or addiction (alcohol, drugs)

### Present Condition:

clinically relevant deviations from normal of any finding during pre-study testing (including blood pressure, pulse and ECG)
any acute disease, especially virus diseases e.g. infectious mononucleosis
any chronic disease likely to modify absorption, metabolism or excretion of the drug under investigation
weight not within ±15% of normal for height, sex and frame as defined by the Modified Metropolitan Life Insurance Company Tables 1983
inability to participate in the entire trial period
participation in a clinical investigation within 30 days prior to first drug administration
use of medication within 14 days before first administration, or within less than 10 times of elimination half life of the respective drug, if not expressively allowed by sponsor (except hormonal contraceptives in females)
blood donation of more than 500 mL within the last 30 days and intention to blood donation within 30 day after study end, or difficulty in donating blood
tobacco use (>10 cigarettes per day) in the 3 months prior to study dosing
use of alcohol or xanthine or grapefruit containing food or drink for 24 hours prior to the drug administration of each period
consumption of “abnormal” quantities of alcohol, coffee, tea or tobacco as judged by the investigator
no reliable contraception (reliable contraception: hormonal contraceptives (must have been taken consistently for at least one month prior to receiving study medication), IUD, sterilization) (females only)
pregnancy or breast-feeding period (females only)
supine blood pressure: >150/95 mmHg

### 4.1.2 Treatments Administered

Subjects received two (2) products by two (2) different modes of administration during the study, with a washout of two (2) weeks between doses. Drug administration took place in the morning with appropriate intervals between subjects to allow for sample collection and any necessary additional clinical observations.

The respective treatments of the two propoxyphene napsylate products were listed in Table 14 below:

TABLE 14

| | | | Total Dose of | |
|---|---|---|---|---|
| | Investigational | Description of | Propoxyphene | Total Dose of |
| Treatment | Product | Investigational Product | Napsylate | Acetaminophen |
| B | Test | Propoxyphene napsylate APAP | 200 mg per | 1,300 mg per |
| | | Modified Release | period | period |
| | | 100 mg propoxyphene napsylate/ | | |
| | | 650 mg acetaminophen | | |
| | | (2 tablets; two at 0 h) | | |
| C | Reference | Darvocet-N 100 ® | 200 mg per | 1,300 mg per |
| | | 100 mg propoxyphene napsylate/ | period | period |
| | | 650 mg acetaminophen | | |
| | | (2 tablets; one at 0 h and one at | | |
| | | 4 h) | | |

B Propoxyphene napsylate APAP Modified Release tablet (fasted dose)
C: Darvocet-N 100 ®, Immediate Release marketed tablet (fasted dose)

The solid dosage forms were administered orally with 240 mL water. The study medication was in individual vials and was administered orally directly from the vial. All oral drug administrations were performed, in accordance with the specifications of the investigator. This included checking the oral and buccal cavity with the aid of a flashlight and tongue depressor. The time of drug administration was considered as study time 0 d0 h in each period.

### 4.1.3 Selection of Doses in the Study

Propoxyphene napsylate APAP Modified release contains 100 mg of propoxyphene napsylate (40 mg in immediate-release form, the rest as extended-release) and 650 mg of acetaminophen per tablet (formulated for approximately 50% to release immediately, remainder as extended release) formulated to be dosed as tablet every 8 hours. Darvocet-N 100® Tablets are formulated as 100 mg of propoxyphene napsylate and 650 mg of acetaminophen in an immediate release formulation releasing in the stomach. Peak concentrations of propoxyphene are reached in 1 to 3 hours and within 2 hours for acetaminophen. After administration of 100 mg propoxyphene napsylate, peak plasma levels of 0.05 to 0.1 μg/mL are achieved. Acetaminophen peak plasma levels after a 650 mg dose are between 5.0 and 12.0 μg/mL. Propoxyphene has an early half-life of 6-12 hours with a terminal half-life of approximately 20 hours. The half-life of acetaminophen is between 2.0 to 8.5 hours. The Darvocet-N 100® marketed product is dosed as one tablet every four hours, not to exceed 600 mg of propoxyphene napsylate per 24 hour period. The Propoxyphene napsylate APAP modified release should be given as a daily dosage of propoxyphene napsylate comparable to that of the current marketed Darvocet-N 100® product, but with a more convenient dosing (every 8 hours instead of 4 hours).

### 4.1.4 Selection and Timing of Dose for Each Subject

Each subject received single oral two-tablet doses of Propoxyphene napsylate APAP modified release tablets (200 mg propoxyphene napsylate/1300 mg acetaminophen in each two-tablet dose) either after an overnight fast or in another period of the study the subject received two single oral doses of Darvocet-N 100® tablets, (100 mg propoxyphene napsylate/650 mg acetaminophen per tablet) at 0 h and 4 h after an overnight fast. Drug administrations were performed in the next morning after admission to the study unit. The day of drug administration was defined as Day 0. At the completion of the study, each subject received test product under fasted and reference product under fasted condition.

### Relative Dosing Times for Treatments B and C:

| | 0 h | 4 h |
|---|---|---|
| Day 0: | B, C | C |

Treatment B=two Propoxyphene napsylate APAP, modified release tablets, 100 mg propoxyphene napsylate/650 mg acetaminophen in each tablet (200 mg/1300 mg in two-tablet dose). (Test drug)-fasted dose
Treatment C=one Darvocet-N 100® immediate release tablet, 100 mg propoxyphene napsylate/650 mg acetaminophen (Reference drug)-fasted dose

### Analysis of Pharmacokinetics

Two subjects (Subjects 33 and 36) dropped out after Period 1, two more (Subjects 14, and 15) dropped out after Period 2. Thus, the pharmacokinetic population for all intra-individual evaluations was n=50.

For the evaluation of the plasma concentrations of the three analytes, blood samples withdrawn at 0 (priorto administration), 0.5, 1, 1.5, 2, 2.5, 3, 4, 4.5, 5, 5.5, 6, 7, 8, 10, 12, 15, 18, 21, 24, 36, 48, 72, 96 and 120 hours post dose were used.

### 4.1.4.1 Propoxyphene

All pre-dose samples (0 d0 h) of propoxyphene were below lower limit of quantitation (LLOQ).

Following administration of the second dose of the IR tablet to fasted subjects, plasma concentrations above LLOQ or trough were observed in almost all subjects about one hour post dose. Peak plasma concentrations were reached regularly in most individuals between two and three hours after administration.

Terminal half life and Ke was determined reliably in almost all subjects following all treatments: geometric mean or median about 28 hours.

In comparison to the regular tablet the modified release (ER) preparation administered to fasted subjects revealed a different plasma level profile. Absorption appeared to start similarly as judged by observation of measurable plasma concentrations of propoxyphene in all subjects at one hour post dose. Cmax however was reached much later (median: 8 h). The mean curve is a good representation of most individual profiles. Due to the plateau-like shape, neither tₘₐₓ nor Cₘₐₓ were suited for a detailed statistical comparison.

AUC, however, and the distribution of area along the time axis are robust and reliable parameters to assess relative bioavailability. Both AUC metrics resulted in the same point estimate of the ratio B/C (1.13) with narrow confidence intervals. Both preparations were equivalent with respect to their extent of absorption.

It is obvious however that the ER formulation delays drug release, as tₘₐₓ (on average or in most individuals) of propoxyphene is shifted to later time periods. As a consequence, individual Cₘₐₓ is be lower than an IR preparation of the same strength, and the AUC is similar. In fact, with Treatment C, the IR preparation of the same strength, but administered in two portions four hours apart, the Cₘₐₓ of the modified release form is lower in about 80% of individual subjects.

### 4.1.4.2 Acetaminophen

All pre-dose samples of acetaminophen were below LLOQ.

Following administration of the second dose of the IR tablet to fasted subjects, plasma concentrations of acetaminophen above LLOQ are observed in almost all subjects about 30 min post dose. Peak plasma concentrations are reached regularly in most individuals between one and two hours after administration.

Terminal half life and Ke of acetaminophen could be determined reliably in almost all subjects following all treatments with the geometric mean or median of 4.5 to 5.5 hours.

In comparison to the regular tablet, the ER preparation administered to fasted subjects revealed a different plasma level profile. Absorption appeared to start similarly fast as judged by observation of measurable plasma concentrations of acetaminophen in all subjects at 30 min post dose. Cₘₐₓ, however was reached later (median: 3 h). The mean curve is a good representation of most individual profiles. Due to the plateau-like shape neither tₘₐₓ nor Cₘₐₓ are suited for a detailed statistical comparison.

AUC however, and the distribution of area along the time axis are robust and reliable parameters to assess relative bioavailability of acetaminophen. Both AUC metrics for acetaminophen resulted in the same point estimate of the ratio B/C (0.96) with very narrow confidence intervals. Both preparations are equivalent with respect to their extent of absorption of acetaminophen.

It is obvious however that the ER formulation is an excellent modified release form, as tₘₐₓ (on average or in most individuals) of acetaminophen is shifted to later time periods. As a consequence, individual Cₘₐₓ must be lower than with an IR preparation of the same strength.

### 4.1.5 Statistical/Analytical Issues

TABLE 15

| | | | |
|---|---|---|---|
| B/C | propoxyphene | 113 | 114 |
| (MR fasted/ | | (108–119) | (108–120) |
| IR fasted) | acetaminophen | 96 | 95 |
| | | (93–99) | (92–99) |

* Pratt-Wilcoxon test and nonparametric confidence intervals according to Tukey

For the following statements it has to be kept in mind that Cₘₐₓ and tₘₐₓ of modified release formulations (ER) are bound to great variations due to the expected plateau and should be interpreted with great caution. The same arguments apply to the resulting ratios.

Overall exposure as measured by AUC(0-∞) or AUC(0-t_{z}) of the ER in comparison to IR under fasted conditions (B/C) was slightly increased for propoxyphene and slightly reduced for acetaminophen. The confidence intervals are all narrow and well within the usual acceptance limits of 80% to 125% commonly used to demonstrate bioequivalence.

### 4.1.6 Pharmacokinetic Conclusions

Relative bioavailability of propoxyphene and acetaminophen was assessed for a new modified release preparation (Treatment B) in comparison to the regular (instant release) tablet (Treatment C, reference) in fasted subjects (n=50). AUC metrics for propoxyphene resulted in an estimate of the ratio B/C (1.13) with narrow confidence intervals (1.08, 1.19). AUC metrics for acetaminophen resulted in an estimate of the ratio B/C (0.96) with narrow confidence intervals (0.93, 0.99). Both treatments are equivalent with respect to their exposure or the extent of absorption, for both active drug ingredients.

For this single dose comparison of two preparations with different release characteristics no formal assessment of rate aspects of availability was attempted.

### 5 Discussion and Overall Conclusions

54 subjects were treated with study medications according to protocol. Two subjects left the trial after the second period and two subjects were withdrawn from the study at the time of check-in in period 2 due to positive urine drug or alcohol breath tests. The remaining 50 subjects were dosed and also completed the trial as planned. Full data sets are available for 50 subjects.

Relative bioavailability of propoxyphene and acetaminophen was assessed for a new modified release preparation (Treatment B) in comparison to the regular (instant release) tablet (Treatment C, reference) in fasted subjects (n=50). AUC metrics for propoxyphene resulted in an estimate of the ratio B/C (1.13) with narrow confidence intervals (1.08, 1.19). AUC for acetaminophen resulted in an estimate of the ratio B/C (0.96) with narrow confidence intervals (0.93, 0.99). Both treatments are equivalent with respect to exposure for both active drug ingredients.

For this s.d. comparison of two preparations with different release characteristics no formal assessment of rate aspects of availability was attempted.

### EXAMPLE 16

This example provides a summary of the Clinical Study Report for an open-label, randomized, multiple dose, two-way cross-over, comparative, bioavailability study in healthy volunteers with an immediate and modified release propoxyphene/acetaminophen (APAP) formulation.
Title: An Open-Label, Randomized, Multiple Dose, Two-Way Cross-Over, Comparative, Bioavailability Study in Healthy Volunteers with an Immediate and Modified Release Propoxyphene/Acetaminophen Formulation
Sponsor's study code: XP20B-102
EduraCT number: 2005-004592-37
AAI Deutschland study code: PA400
Phase: Phase I
Name of the investigational products: Test drug: Propoxyphene Napsylate APAP Modified Tablets Reference drug: Darvocet-N 100® Tablets

### 7 Synopsis

### Title of Study:

An Open-Label, Randomized, Multiple Dose, Two-Way Cross-Over, Comparative, Bioavailability Study in Healthy Volunteers with an Immediate and Modified Release Propoxyphene/Acetaminophen Formulation

### Objective:

To assess the steady state bioavailability bioavailability of propoxyphene and acetaminophen (APAP) between the test drug (Propoxyphene Napsylate APAP Modified Release tablets) and the reference drug (Darvocet N-100 Immediate Release tablets) evaluated under fasted conditions.

### Methodology:

A multiple-dose, open label, randomized, two-way crossover, relative bioavailability study in healthy volunteers.

### Number of Subjects (Planned and Analyzed):

| | |
|---|---|
| Number of subjects planned: | 86 subjects |
| Number of subjects included: | 87 subjects |
| | 37 females |
| | 50 males |
| Number of subjects analyzed: pharmacokinetic data: | 74 subjects |
| | 28 females |
| | 46 males |

### Diagnosis and Main Criteria for Inclusion:

Healthy light smoking (≦10 cigarettes/day) and non-smoking, male and female volunteers of Caucasian race between 18 and 50 years of age, within −5%, +20% of normal body weight for height, frame and gender and body weight at least 65 kg.

### Test Drug, Dose and Mode of Administration, Batch Number:

Propoxyphene Napsylate modified release (R tablet, 100 mg propoxyphene napsylate/650 mg acetaminophen, batch number of manufacturer: K05068E, administered as multiple oral doses every eight hours for a total of 28 two-tablet doses (Treatment A).

### Duration of Treatment:

Multiple oral doses as one tablet every four hours for a total of 56 doses of Darvocet-N 100® (100 mg propoxyphene napsylate/650 mg of acetaminophen) in one period of the study and multiple oral doses every eight hours for a total of 28 two-tablet doses of Propoxyphene napsylate APAP modified release tablets (200 mg propoxyphene napsylate/1300 mg acetaminophen in each two-tablet dose) in another period of the study separated by between-treatment intervals of 16 or 19 days.

Reference Drug, Dose and Mode of Administration, Batch Number: Darvocet-N 100®, immediate release (IR) tablet, 100 mg propoxyphene napsylate/650 mg acetaminophen administered as multiple oral doses of one tablet every four hours for a total of 56 doses (Treatment B).
Pharmacokinetics: Pharmacokinetic parameters were calculated for acetaminophen and propoyphene in plasma. Blood samples per period were collected at the following times: 5 d0 h, 6 d0 h, 7 d0 h, 8 d0 h, 9 d0 h, 9 d0.5 h, 9 d1 h, 9 d1.5 h, 9 d2 h, 9 d3 h, 9 d4 h, 9 d4.5 h, 9 d5 h, 9 d5.5 h, 9 d6 h, 9 d7 h, and 9 d8 h post-dose.
Primary variables: AUCₛₛ=area under the concentration time curve calculated by the linear trapezoidal rule (time 0 to 8 hours at steady state)

Cₘₐₓ=measured maximum concentration during a nominal dosage interval (8 hours) at steady state, primarily as metric of exposure
Secondary variables: tₘₐₓ=time of observed plasma concentration maximum

(Cₘₐₓ) during a nominal dosage interval (8 hours) at steady state.

Cₐᵥₑ=average concentration during steady state, AUCₛₛ/8 h

Cₘᵢₙ=measured minimum concentration during a nominal dosage interval (8 hours) at steady state

PTF%=Percent peak-trough fluctuation at steady state, 100% (Cₘₐₓ-Cₘᵢₙ)/Cₐᵥₑ

### Pharmacokinetic Results

The main pharmacokinetic conclusions are as follows:
Equivalence in relative bioavailability, assessed by AUCₛₛ, the primary variable, was established for the modified release as compared to the immediate release form of Darvocet-N for propoxyphene and acetaminophen at steady state (nominal dose interval 8 h).
The fluctuation in plasma levels of acetaminophen is reduced for the modified release form with the minimum concentrations remaining on the same level as the immediate release tablets.
There are no remarkable differences in Cₘₐₓ and Cₘᵢₙ between modified and immediate release form with regard to propoxyphene.
The ER formulation (administered every 8 hours) is an excellent modified release form with respect to both active ingredients, and the formulation is essentially equivalent to the currently marketed IR version.

TABLE 16
Results of the Modified Release vs. Immediate Release Tablets
Ratios of Least Squares Means (90% Confidence Intervals)

| | | Pharmacokinetic Parameters, Geom. Means | | | |
|---|---|---|---|---|---|
| Ratio [%] | | (CI) | | | |
| (90% CI) | Ingredient/Analyte | °AUCₛₛ | °Cₘₐₓ | °Cₘᵢₙ | °PTF %* |
| Test/Reference | propoxyphene | 101 | 98 | 102 | 43.4%/49.7% |
| (ER: 2 tablets | | (97–105) | (93–103) | (98–107) | |
| every 8 hours/ | acetaminophen | 90 | 72 | 104 | 104%/151% |
| IR: 1 tablet | | (85–94) | (67–77) | (97–111) | |
| every 4 | | | | | |
| hours) | | | | | |

geom.means test/geom. means reference
°at steady state in the nominal dose interval of 8 h

### Conclusion:

Eighty seven (87) subjects were included in this study. The study was performed according to the study protocol. The AUCₛₛ in the nominal dose interval of 8 h was used to describe the extent of drug absorption and as basis for determination of the relative bioavailability by statistical methods. Equivalence in relative bioavailability (assessed by the primary variable) was shown for the modified release as compared to the immediate release form of Darvocet-N for propoxyphene and acetaminophen. The fluctuation in plasma levels of acetaminophen is reduced for the modified release form with the minimum concentrations remaining on the same level as the immediate release tablets. There are no remarkable differences in Cₘₐₓ and Cₘᵢₙ between modified and immediate release form with regard to propoxyphene. The MR formulation (administered every 8 hours) is an excellent modified release form with respect to both active ingredients. In addition, both treatments are equivalent in terms of their relative bioavailability, bearing in mind that the reference drug (IR tablets) has the inherent timing caused by the dosing schedule (every 4 hours).

### 7.1 Rationale

There were no in vitro tests that were conclusive in assessing the bioavailability of propoxyphene/acetaminophen combination preparations. Therefore, a pharmacokinetic comparison study in human subjects had to be performed. A new modified form of propoxyphene napsylate APAP that contains 100 mg of propoxyphene napsylate (40 mg in immediate-release form, 60% as extended-release) and 650 mg of acetaminophen (formulated for approximately 50% to release immediately, 50% as extended release) was developed. The total daily dose of both the modified and currently marketed Darvocet-N 100® product was expected to be comparable. The modified release tablet, with its extended-release propoxyphene napsylate and acetaminophen components, was designed to be dosed as two tablets every 8 hours, instead of one tablet every 4 hours as is the currently marketed product. The goal is to provide more convenient dosing (i.e., dosed less frequently) which produces less peak to trough plasma concentration fluctuation over the dosing interval while providing comparable total exposure and less or comparable peak exposure to the active durg ingredients (and metabolites).

Combinations of an opioid for weak and moderate pain and a non opioid analgesic are recommended by WHO for the treatment of moderate to severe pain stage 2. They recognize the use of these combinations for treating pain in subjects who do not require a strong opioid, but are not free of pain after treatment with NSAIDs or non-opioid analgesics alone. Combination therapy is a generally recognized medical treatment modality. By combining two different analgesics into one preparation, one expects an addition of the analgesic efficacy at the lowest possible dose of the single active agents. Combinations of a mild opioid and NSAIDs or acetaminophen are well accepted treatment modality.

The present study was designed as an open-label, randomized, multiple dose, and explorative bioavailability evaluation of two different propoxyphene napsylate combination tablets at steady state conditions in 86 healthy volunteers. Dose interval was chosen to match the recommended use with 4 h for the currently marketed product and 8 h for the new modified release form, but the daily dose of all active ingredients was identical.

### 8. Study Objectives

The primary objective of this study was to evaluate the comparative bioavailability of the modified release formulation to that of the marketed immediate release tablets at steady state.

### Primary Variables to be Studied in Detail Were:

Area under the curve (AUCₛₛ) and maximum concentration (Cₘₐₓ)

### Secondary Variables to be Studied Were:

Time of maximum (tₘₐₓ), percent fluctuation peak-to-trough (PTF%), minimum concentration (Cₘᵢₙ), and average concentration during steady state (Cₐᵥₑ).

### Dose Considerations:

The dose level was chosen to cover the full daily dose permitted. These conditions were expected to approach the tolerability limit of healthy volunteers and a pharmacokinetic response possibly outside the dose proportional range in some subjects. These conditions do have some influence on the variables that were measured.

### Pharmacokinetic Considerations:

The study conditions were chosen to optimize the assessment of AUCₛₛ, the primary variable for extent of absorption. Cₘₐₓ has to be considered in this setting as a measurement of exposure, rather than to characterize the rate aspect of availability. Time of maximum (tₘₐₓ) has to be considered in conjunction with Cₘₐₓ, in order to be useful to describe rate of availability. Values of tmax were assessed for the dose interval 8 h, in order to characterize the modified release formulation, but not to be compared between forms (instant or modified release) with differing dose intervals. PTF% and Cₘᵢₙ are measurements that are dependent on the dose interval. They can be compared in their magnitude, as they characterize the pharmacokinetic properties of the pharmaceutical dosage form under the chosen (comparable) mode of administration. Cₘᵢₙ is a useful metric of exposure similar to Cₘₐₓ, and PTF% thus a measure of relative exposure. Formally, Cₘₐₓ and Cₘᵢₙ are not covered by acceptance ranges in testing guidelines.

### 7.1.1 Overall Study Design

In this open-label, randomized, crossover, multiple dose, two-way cross-over, comparative, steady state bioavailability study in 87 healthy volunteers, the propoxyphene, norpropoxyphene and acetaminophen pharmacokinetic parameters of Propoxyphene napsylate APAP modified release tablets and Darvocet-N 100® tablets were to be evaluated and compared statistically. The study was performed under in-patient conditions with eighty seven (87) healthy light smoking (≦10 cigarettes/day) and non-smoking, male and female volunteers of Caucasian race between 18 and 50 years of age.

In each period, the subjects were administered one Darvocet-N 100® tablet every four hours for 9 days followed by doses at 0 and 4 hours on the 10^{th} day (Day 9), or two Propoxyphene napsylate APAP modified release tablets every eight hours for 9 days followed by a single two-tablets morning dose on the 10^{th} day (Day 9). A total of 216 hours of dose administration (9 complete days) was required in order to have all analytes reach steady state conditions. The sequence of treatment administration (test-then-reference or reference-then-test) was randomized so that an equal number of subjects were in each dosing sequence. The washout phase between the two (2) periods was sixteen (16) or nineteen (19) days.

The study was performed in two consecutive groups with approximately equal size of subjects.

### 7.1.1.1 Dietary Regimen

The subjects were fasting from at least 10 hours before the 0 hour dose on dosing 0 d0 h (Day 0) and 9 d0 h (Day 9), until a light breakfast was served at 2 hours. Water intake was ad lib except 1 hour pre dose to 1 hour post dose. Six (6) hours after the 0 hour dose on each dosing day, a standardized lunch was served.

All other meals were served so as not to be within 2 hours of dosing. To allow adequate time for meal service, times stated were ±15 minutes. When the times for a meal and a blood sample coincide, the blood sample was collected as scheduled, prior to the meal.

Starting 24 hours before first medication of each period and during the confinement periods, alcohol, xanthine or grapefruit containing beverages and food as coffee, tea, cocoa, chocolate, nuts, coke, etc., were not allowed to be consumed.

### Inclusion Criteria

Healthy light smoking (≦10 cigarettes/day) and non-smoking, male and female volunteers of Caucasian race between 18 and 50 years of age were recruited from the local population and eighty six (87) eligible subjects (37 females and 50 males) were included in the study after having given voluntary written informed consent before the first invasive pre-study examination procedure.

### Exclusion Criteria

History of:
drug hypersensitivity to propoxyphene, codeine, acetaminophen or similar NSAID compounds
seizure disorder
severe digestive disorder
surgery of the digestive tract (except for appendectomy)
liver function disturbances
renal disorders (albuminuria, chronic infections)
respiratory or cardiovascular disorders
diabetes mellitus
hyperthyroidism or other endocrine disorders
malignancy
recent history of mental illness
history of substance abuse or addiction (alcohol, drugs)

### Present Condition:

clinically relevant deviations from normal of any finding during pre-study testing (including blood pressure, pulse and ECG)
any acute disease, especially virus diseases e.g. infectious mononucleosis
any chronic disease likely to modify absorption, metabolism or excretion of the drug under investigation
weight not within −5%, +20% of normal for height, sex and frame as defined by the Modified Metropolitan Life Insurance Company Tables 1983 and body weight lower than 65 kg
inability to participate in the entire trial period
participation in a clinical investigation within 30 days prior to first drug administration
use of medication within 14 days before first administration, or within less than 10 times of elimination half life of the respective drug, if not expressively allowed by Sponsor (except hormonal contraceptives in females)
blood donation of more than 500 mL within the last 30 days or difficulty in donating blood
tobacco use (>10 cigarettes per day) in the 3 months prior to study dosing
use of alcohol or xanthine or grapefruit containing food or drink for 24 hours prior to the drug administration of each period.
consumption of “abnormal” quantities of alcohol, coffee, tea or tobacco as judged by the investigator
no reliable contraception (reliable contraception: hormonal contraceptives (must have been taken consistently for at least one month prior to receiving study medication), IUD, sterilization) (females only)
pregnancy or breast-feeding period (females only)
supine blood pressure: >150/95 mmHg

### 7.1.2 Treatments Administered

Subjects received two (2) treatments (multiple doses of the test treatment: 600 mg propoxyphene napsylate/3,900 mg acetaminophen per day for nine days and 200 mg/1,300 mg on the 10^{th} day, and multiple doses of the reference treatment: 600 mg propoxyphene napsylate/3,900 mg acetaminophen per day for nine days and 200 mg/1,300 mg on the 10^{th} day) during the study, with a washout phase of sixteen (16) or nineteen (19) days. Drug administrations started at Day 0 with appropriate intervals between subjects to allow for sample collection and any necessary additional clinical observations. The respective treatments consisted of the following as shown in Table 17:

TABLE 17

| | | | Total Dose of | |
|---|---|---|---|---|
| | Investigational | Description of | Propoxyphene | Total Dose of |
| Treatment | Product | Investigational Product | Napsylate | Acetaminophen |
| A | Test | Propoxyphene Napsylate | 600 mg per day for | 3,900 mg per day |
| | | APAP Modified Release | nine days and | for nine days and |
| | | 100 mg propoxyphene | 200 mg on the 10^{th} | 1,300 mg on the |
| | | napsylate/650 mg | day | 10^{th} day |
| | | acetaminophen | | |
| | | (2 tablets every 8 h) | | |
| B | Reference | Darvocet-N 100 ® | 600 mg per day for | 3,900 mg per day |
| | | 100 mg propoxyphene | nine days and | for nine days and |
| | | napsylate/650 mg | 200 mg on the 10^{th} | 1,300 mg on the |
| | | acetaminophen | day | 10^{th} day |
| | | (1 tablet every 4 h) | | |

The solid dosage forms were administered orally directly from the vial with 240 mL water. The time of first drug administration was considered as study time 0 d0 h.

### 7.1.3 Selection of Doses in the Study

Propoxyphene napsylate APAP modified release contains 100 mg of propoxyphene napsylate (40 mg in immediate-release form, the rest as extended-release) and 650 mg of acetaminophen per tablet (formulated for approximately 50% to release immediately, remainder as extended release) formulated to be dosed as one to two tablets every 8 hours. Darvocet-N 100® Tablets are formulated as 100 mg of propoxyphene napsylate and 650 mg of acetaminophen in an immediate release formulation releasing in the stomach. Peak concentrations of propoxyphene are reached in 1 to 3 hours and within 2 hours for acetaminophen. After administration of 100 mg propoxyphene napsylate, peak plasma levels of 0.05 to 0.1 μg/mL are achieved. Acetaminophen peak plasma levels after a 650 mg dose are between 5.0 and 12.0 μg/mL. Propoxyphene has an early half-life of 6-12 hours with a terminal half-life of approximately 20 hours. The half-life of acetaminophen is between 2.0 to 8.5 hours. The Darvocet-N 100® marketed product was dosed as one tablet every four hours, not to exceed 600 mg of propoxyphene napsylate per 24 hour period. The Propoxyphene napsylate APAP modified release was given as a daily dosage of propoxyphene napsylate comparable to that of the current marketed Darvocet-N 100® product, but with a more convenient dosing (every 8 hours instead of 4 hours).

### 7.1.4 Selection and Timing of Dose for Each Subject

Each subject received multiple oral doses as one tablet every four hours for a total of 56 doses of Darvocet-N 100® (100 mg propoxyphene napsylate/650 mg of acetaminophen) in one period of the study and multiple oral doses every eight hours for a total of 28 two-tablet doses of Propoxyphene napsylate APAP modified release tablets (200 mg propoxyphene napsylate/1,300 mg acetaminophen in each two-tablet dose) in another period of the study. Drug administrations began at the following day after admission to the study unit. The day of the first drug administration was defined as Day 0.

TABLE 18
Relative dosing times for Treatment A and Treatment B:

| | | 0 h | 4 h | 8 h | 12 h | 16 h | 20 h |
|---|---|---|---|---|---|---|---|
| Admission-day | Day-1 | X | | | | | |
| 1^{st} dosing day | Day 0: | A, B | B | A, B | B | A, B | B |
| 2^{nd} dosing day | Day 1: | A, B | B | A, B | B | A, B | B |
| 3^{rd} dosing day | Day 2: | A, B | B | A, B | B | A, B | B |
| 4^{th} dosing day | Day 3: | A, B | B | A, B | B | A, B | B |
| 5^{th} dosing day | Day 4: | A, B | B | A, B | B | A, B | B |
| 6^{th} dosing day | Day 5: | A, B | B | A, B | B | A, B | B |
| 7^{th} dosing day | Day 6: | A, B | B | A, B | B | A, B | B |
| 8^{th} dosing day | Day 7: | A, B | B | A, B | B | A, B | B |
| 9^{th} dosing day | Day 8: | A, B | B | A, B | B | A, B | B |
| 10^{th} dosing day | Day 9: | A, B | B | — | — | — | — |

| | | |
|---|---|---|
| 11^{th} day discharge | Day 10 | X |

Treatment A = two Propoxyphene Napsylate APAP Modified Release tablets, 100 mg propoxyphene napsylate/650 mg acetaminophen in each tablet (200 mg/1,300 mg in two-tablet dose). (Test drug)
Treatment B = one Darvocet-N 100 ® Immediate Release tablets, 100 mg propoxyphene napsylate/650 mg acetaminophen (Reference drug)

### 7.1.4.1 Appropriateness of the Pharmacokinetic Variables

The AUCₛₛ in the nominal dose interval of 8 h was used to describe the extent of drug absorption and as basis for determination of the relative bioavailability by statistical methods.

Cₘₐₓ has to be considered in this setting rather as metric of exposure. The rate aspect of absorption is described generally by a combined consideration of Cₘₐₓ and tₘₐₓ due to the lack of more appropriate methods. Considering only a single peak parameter may provide less reliable or sometimes even misleading estimates. With modified release formulations, as investigated in this study, this is often the case. This is in particular true if multiple peaks or plateau-like profiles are observed. In addition, with the statistical evaluation as single parameter, artifacts may occur frequently and arouse suspicion about treatment, period, or sequence effects in one or both of the metrics.

The percent fluctuation is a measure of the consistency of the steady state concentrations within the interval used for calculation and thus not independent of the dose interval. It characterizes the pharmacokinetic properties of the pharmaceutical dosage form under the chosen mode of administration including the dose interval during steady state. Thus, PTF% can be compared in its magnitude between different steady state settings. PTF% is a useful metric for relative exposure.

Cₘᵢₙ in the nominal dose interval of 8 h is a useful metric of exposure similar to Cₘₐₓ. Interpretations of Cₘᵢₙ and PTF% have to be made only in the context of the intended clinical use.

### 7.1.5 Analysis of Pharmacokinetics

Thirteen (13) subjects of 87 dosed dropped out and did not complete the study. Thus, the pharmacokinetic population for all intra-individual evaluations was n=74.

For the evaluation of the plasma concentrations of the three analytes, blood samples were collected at the following times: 0 d0 h (pre-dose), 5 d0 h, 6 d0 h, 7 d0 h, 8 d0 h, 9 d0 h, 9 d0.5 h, 9 d1 h, 9 d1.5 h, 9 d2 h, 9 d3 h, 9 d4 h, 9 d4.5 h, 9 d5 h, 9 d5.5 h, 9 d6 h, 9 d7 h, and 9 d8 h post-dose.

### 7.1.5.1 Propoxyphene

The visual inspection of the data collected from Day 5 up to Day 9 with both treatments and the corresponding plots indicate that steady-state conditions have sufficiently been achieved at Day 9 in all individual subjects.

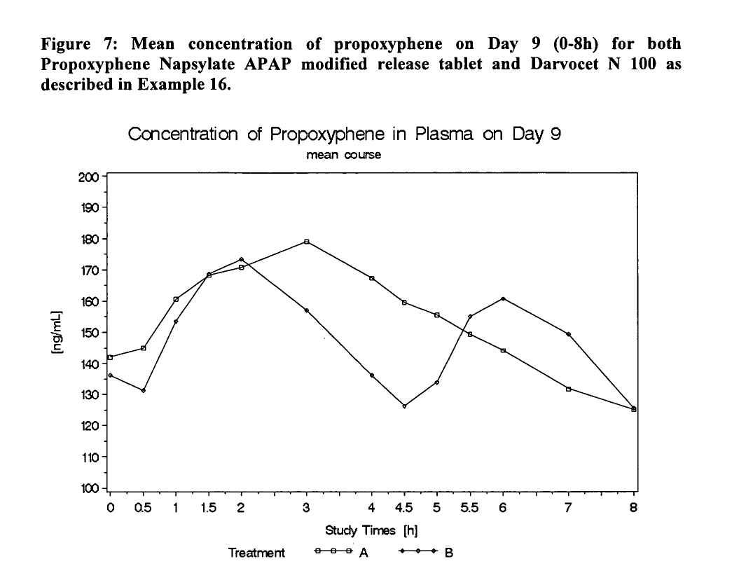

The mean course of concentration of propoxyphene following the modified release preparations (modified release (ER), test) and the immediate release tablets (IR, reference) on Day 9 (0-8 h) is given in FIG. 7. Each mean curve is a good representation of most individual profiles.

As was expected, the modified release form (2 tablets every 8 hours) revealed a different plasma level profile in comparison to the immediate release form (1 tablet every 4 hours) which tends to form two peaks within the 8 h interval. Therefore, assessments for tₘₐₓ were not comparable between the two treatment groups.

AUCₛₛ in the steady state dose interval of 8 h, however, is a reliable parameter to assess relative bioavailibility whereas Cₘₐₓ, Cₘᵢₙ and PTF% have been evaluated primarily in order to assess potential differences with regard to the fluctuation of plasma levels.

The AUC ratio of 101%. (CI: 97%-105%) shows equivalence in bioavailibility for the modified release as compared to the immediate release form.

The fluctuation of plasma levels is slightly reduced for the modified release form (PTF of 43.4% vs. 49.7% for IR). The corresponding values for Cₘₐₓ (175.6 ng/mL for ER vs. 179.9 ng/mL for IR) and Cₘᵢₙ (109.3 ng/mL for ER vs. 106.8 ng/mL for IR) do not show any remarkable differences. For Cₘₐₓ, Cₘᵢₙ and PTF% ratios.

Hence, the effect of the modified release can be regarded as smoothing the concentration-time curve in comparison to immediate release, but without concomitant loss of bioavailability with regard to propoxyphene.

### 7.1.5.2 Acetaminophen

All pre-dose samples of acetaminophen were below LLOQ (0.1 μg/mL).

The visual inspection of the data collected from Day 5 up to Day 9 and the corresponding plots indicate that steady-state conditions were achieved at Day 9 with both treatments in all individual subjects.

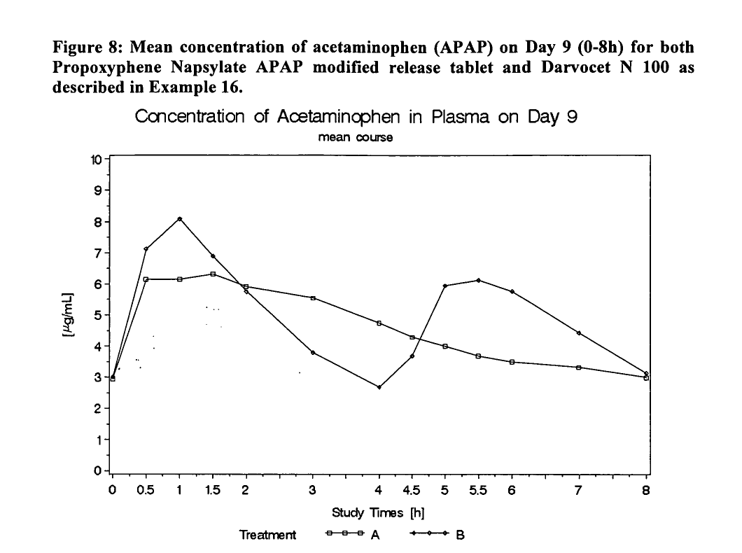

The mean course of concentration of acetaminophen on Day 9 (0-8 h) is given for both treatments in FIG. 8. Each mean curve is a good representation of most individual profiles.

As was expected, the modified release form (2 tablets every 8 hours) revealed a different plasma level profile in comparison to the immediate release form (1 tablet every 4 hours) which tends to form two peaks within the 8 h interval. Therefore, assessments for tₘₐₓ are not comparable between the two treatment groups.

AUCₛₛ in the steady state dose interval of 8 h, however, is a reliable parameter to assess relative bioavailability, whereas Cₘₐₓ, Cₘᵢₙ and PTF% have been evaluated primarily to assess potential differences with regard to exposure and to the fluctuation of plasma levels.

The AUC ratio of 90% (CI: 85%-94%) shows equivalence in bioavailibility with respect to acetaminophen for the modified release as compared to the immediate release form according to all commonly accepted criteria. The relative bioavailibity of the modified release form tends to be a somewhat below unity, a fact that is illustrated by the statistical significance of treatment differences for acetaminophen.

The fluctuation of plasma levels is cleary less pronounced after the modified release form (PTF of 104.4% vs. 150.5% for the IR). Peak concentrations (Cₘₐₓ) are lower (6.83 μg/mL vs. 9.52 μg/mL for IR). Minimal exposure at steady state (Cₘᵢₙ, 2.17 μg/mL for ER vs. 2.09 μg/mL for IR) does not show any difference. Accordingly in 57% of the subjects, Cₘᵢₙ of ER exceeds that after IR. For Cₘₐₓ, Cₘᵢₙ and PTF% ratios.

Hence, the effect of the modified release (ER) can be regarded as smoothing the concentration-time curve by reducing the maximum concentrations, whereas no reduction in minimum concentrations can be observed. Exposure in terms of AUC or in terms of Cₘᵢₙ is comparable between both treatments. In that regard, all requirements necessary to conclude equivalence between both preparations were fulfilled.

### 7.1.5.3 Pharmacokinetic Characteristics of the Pharmaceutical Preparations

All analytes contribute to characterize certain properties of the combination tablets, both the immediate and the modified release form. The most direct information comes from the ingredients monitored. This is at variance to the biological activity or biological equivalence, which in this respect, the data of the active metabolite have their distinct value.

The inter-individual variability with respect to the key kinetic parameters at steady state is highly comparable between both treatments, as evident from Table 19. The fact that the modification in the release does not increase variability in the pharmacokinetic response contributes to the favorable properties of the MR tablets.

### Table 19: Variability in Key Pharmacokinetic Parameters at Steady State (%CV, geo)

| Analyte | Form | AUCₛₛ | Cₘₐₓ | Cₘᵢₙ | Cₐᵥₑ | % PTF* |
|---|---|---|---|---|---|---|
| Propoxyphene | ER | 43 | 43 | 43 | 43 | 43 |
| Propoxyphene | IR | 37 | 38 | 40 | 37 | 50 |
| Acetaminophen | ER | 38 | 37 | 43 | 38 | 105 |
| Acetaminophen | IR | 30 | 27 | 44 | 30 | 151 |

*as % PTF is already a measure of variability, it is given directly and not as % CV, geo

The low variability is also manifested in the observation that mean curves of all treatments and all analytes are representative for most individuals.

The study conditions were chosen to optimize the assessment of AUCSₛₛ, the primary variable for extent of absorption and exposure. For this steady state comparison of two preparations with different release characteristics and differing dose frequencies no formal assessment of rate aspects of availability was attempted. Cₘₐₓ (and Cₘᵢₙ or Cₐᵥₑ) have to be considered in this setting rather as metric of exposure. All measures of exposure together (including %PTF), are well suited for an evaluation of the performance of the new modified release tablet in a steady state interval.

With these criteria in mind, it is obvious that the ER formulation is an excellent modified release form. But meaningful interpretations of Cₘᵢₙ, Cₘₐₓ, and PTF% have to be made only in the context of the intended clinical use.

### 7.1.5.4 Statistical/Analytical Issues

The ratios for the relevant pharmacokinetic parameters are given in Table 20.

TABLE 20
Ratios of Pharmacokinetic Parameters

| Ratio [%] | | Pharmacokinetic Parameters, Geom. Means (CI) | | | |
|---|---|---|---|---|---|
| (90% CI) | Ingredient/Analyte | °AUCₛₛ | °Cₘₐₓ | °Cₘᵢₙ | °PTF %* |
| Test/Reference | propoxyphene | 101 | 98 | 102 | 43.4%/ |
| (MR: 2 tablets | | (97–105) | (93–103) | (98–107) | 49.7% |
| every 8 hours/ | acetaminophen | 90 | 72 | 104 | 104%/ |
| IR: 1 tablet | | (85–94) | (67–77) | (97–111) | 151% |
| every 4 hours) | | | | | |

*geom. means test/geom. means reference
°at steady state in the nominal dose interval of 8 h

Equivalence in bioavailability, assessed by AUCₛₛ in the steady state dose interval of 8 h for the modified release as compared to the immediate release form of Darvocet-N could be shown for propoxyphene and acetaminophen.

The relative bioavailibity with regard to propoxyphene and norpropoxphene is perfectly close to an AUC ratio of 1. For acetaminophen, the 90% confidence interval for AUCₛₛ indicates a lower relative bioavailability for the modified release form; however, the interval clearly meets the commonly accepted equivalence criteria, as its lower limit is distinctly higher than the reference value of 80%.

Due to the different administration profiles of the treatments, the results for Cₘₐₓ, Cₘᵢₙ and PTF% is better interpreted with regard to fluctuation or exposure, than in terms of relative bioavailability.

For propoxyphene, the concentration profile of the treatment tend to be contained within borderlines as given by their comparable values of Cₘₐₓ and Cₘᵢₙ. The fluctuation in plasma levels for propoxyphene are comparable, since the differences in PTF% must not be overemphasized, if there are no remarkable differences in Cₘₐₓ and Cₘᵢₙ.

The fluctuations in plasma levels of acetaminophen are, on the other hand, distinctly reduced for the modified release form. This occurs mainly due to a reduction in maximum plasma concentrations. But it is worth noting that the minimum concentrations remain on fairly the same level (even more often with a higher minimum under ER rather than under IR).

### Pharmacokinetic Conclusions

The main pharmacokinetic conclusions are as follows:
Equivalence in relative bioavailibity, assessed by AUCₛₛ—the primary variable, for the modified release as compared to the immediate release form of Darvocet-N could be shown for propoxyphene and acetaminophen at steady state (nominal dose interval 8 h).
The fluctuation in plasma levels of acetaminophen is reduced for the modified release form with the minimum concentrations remaining on the same level as with the immediate release tablets.
There are no remarkable differences in Cₘₐₓ and Cₘᵢₙ between modified and immediate release form with regard to propoxyphene.
It is obvious that the ER formulation (administered every 8 hours) is an excellent modified release form with respect to both active ingredients.
Both treatments are equivalent in terms of their relative bioavailability, bearing in mind that the reference drug (IR tablets) has the inherent timing caused by the dosing schedule (every 4 hours).

### 8 Discussion and Overall Conclusions

87 subjects were included in this study. There were thirteen (13) dropouts due to various reasons as non-adherence to protocol requirements, acute disease, personal reasons, poor tolerability (adverse events). The remaining 74 subjects were dosed and also completed the trial as planned. Full data sets are available for 74 subjects.

Two treatments were compared pharmacokinetically at steady state with respect to relative bioavailability: the modified release tablets (investigational formulation) dosed every eight hours with the reference tablets of immediate release Darvocet-N tablets dosed every four hours. Pharmacokinetic parameters assessed as PTF%, Cₘₐₓ, Cₐᵥₑ, and Cₘᵢₙ are metrics not independent of the dose interval. They can only be compared in their magnitude to characterize the pharmacokinetic properties of the treatment that is the pharmaceutical dosage form under the chosen (comparable) mode of administration. These parameters are to be considered as metrics of exposure or relative exposure in this study.

The AUCₛₛ in the nominal dose interval of 8 h was used to describe the extent of drug absorption and as basis for determination of the relative bioavailability by statistical methods.

Equivalence in relative bioavailability, assessed by the primary variable, for the modified release as compared to the immediate release form of Darvocet-N could be shown for propoxyphene and acetaminophen. The fluctuation in plasma levels of acetaminophen is reduced for the modified release form with the minimum concentrations remaining on the same level as with the immediate release tablets. There are no remarkable differences in Cₘₐₓ and Cₘᵢₙ between modified and immediate release form with regard to propoxyphene. It is obvious that the ER formulation (administered every 8 hours) is an excellent modified release form with respect to both active ingredients, if one bears in mind that the IR tablets have the inherent timing by their dose schedule (every 4 hours), but both treatments are nevertheless equivalent.

### Appendix 1. Definition for abbreviations utilized in Examples 15 and 16.

ANOVA=analysis of variance
AUC₍₀₋ₜ₎=area under the concentration time curve (AUC) calculated from the first sample to time t of last reported value
AUC_{(0-∞)}=area under the concentration time curve (AUC) calculated from time 0 extrapolated to infinity
Autom=Automatic
BA=Bioavailability
BE=Bioequivalence
CFR=code of federal regulations
Cₘₐₓ=maximum concentration
CNS=central nervous system
CRO=contract research organization
DD=drug dictionary
e=Electrophoretic
ECG=Electrocardiogram
EENT=eyes, ears, nose and throat
FDA=Food and Drug Administration
GCMS=gas chromatography mass spectrometry
GCP=Good Clinical Practice
GLM=general linear models
GMP=Good Manufacturing Practice
HPLC=high performance liquid chromatography
IEC=Independent Ethics Committee
IR=Immediate Release
IRB=Institutional Review Board
ISI=International Sensitivity Index
ITT=intent-to-treat
IUD=intrauterine device
i.v.=Intravenous
K=Potassium
Ke=Terminal rate constant
KETON=Ketones
l=Liter
LCMS=liquid chromatography mass spectrometry
LEUCO=Leukocytes
MedDRA=Medical Dictionary for Drug Regulation Affairs
MR=Modified release
MS=mass spectrometry
N=number of observations (count)
Na=Sodium
NC=North Carolina
NITRIT=Nitrites
No=Number
NO=nitric oxide
OPIATE=Opiates
p.a.=After the first dose of drug administration/period
PBC=platelet blood count
pH=hydrogen ion concentration
PIC=Pharmaceutical Inspection Convention
PK=Pharmacokinetics
PR=pulse rate
PREG=pregnancy test
PROT=Protein
QAU=Quality Assurance Unit
RBC=red blood count
SAS=statistical analysis software
SCR=Screening
SOP=standard operating procedure
t½=terminal half-life
tₘₐₓ=time of maximum
U=Urine
UROBIL=Urobilinogen
US=United States
WBC=white blood count
WHO=World Health Organization
XR=Extended Release

### Appendix 2. Single Fasted Pharmacokinetic Variables Used in Example 15.

### Description of the Pharmacokinetic Variables

The following pharmacokinetic parameters were calculated for each subject and each treatment:

### Primary Variables:

AUC(0-∞)=area under the concentration time curve from time 0 extrapolated to infinity calculated by adding C_{z}/λ_{z} to AUC(0-t_{z}).
AUC(0-t_{z})=area under the curve (AUCz) calculated from first sample to time t_{z} (Tz) of last reported value.
Cₘₐₓ=measured maximum concentration

### Secondary Variables:

tₘₐₓ=time of observed maximum
λ_{z} (Ke)=terminal rate constant, by log-linear regression
t½=terminal half-life, calculated as (In 2)/λ₂

The predose sample was always considered as if it had been taken simultaneously with the drug administration. If there were any deviations in sampling, the actual sampling times relative to drug administration was considered if they exceeded the limit specified in Section 9.5.1.2 of this report. Missing data were not replaced or imputed in any way, i.e., they were treated as if the respective sample never had been scheduled. For the calculation of the AUC by the linear trapezoidal rule, this has the same effect as if the missing value had been estimated by linear interpolation. AUC(0-t_{z}) was regarded as unreliable and was not reported if more than two consecutive results were missing or if the concentrations were quantifiable for fewer than 5 time points.

Cₘₐₓ and tₘₐₓ were regarded as unreliable if the maximum was observed preceding or following a sample with missing data. In case of multiple peaks, Cₘₐₓ and tₘₐₓ refered to the highest measured concentration even if there were earlier peaks. In case of two or more samples with the same concentration (as supplied by the analyst), tₘₐₓ refered to the earlier of these.

The data points to be used for calculation of λ_{z} were determined by visual inspection of concentration-time curves in log-linear scaling. The calculation was considered sufficiently reliable in case of a coefficient of determination of R²>0.85 and unreliable in case of R²<0.8. Cases in-between were considered case-by-case. The starting time (T_{z}) for calculation of λ_{z} and the number of data points in the regression (N_{z}) as well as the coefficient of determination R² were also tabulated.

The value of AUC(0-∞) was considered unreliable and was not reported if the terminal area beyond the last quantified sample was greater than 20% of the total AUC(0-∞).

### Appropriateness of the Pharmacokinetic Variables

The AUC is used to describe the extent of drug absorption. The rate of absorption is measured by Cₘₐₓ and tₘₐₓ, if considered in combination. Ke and t_{1/2}, describe the kinetics in the terminal phase, which, for many substances, is governed by elimination processes.

### Appendix 3. Multiple Dose Fasted Pharmacokinetic Variables Used in Example 16.

### Descriptoin of the Pharmacokinetic Variables

The following pharmacokinetic parameters were calculated for each subject and each treatment: Primary variables:
AUCₛₛ=area under the concentration time curve calculated by the linear trapezoidal rule (time 0 to 8 hours at steady state)
Cₘₐₓ=measured maximum concentration during a nominal dosage interval (8 hours) at steady state, primarily as metric of exposure

### Secondary Variables:

tₘₐₓ=time of observed plasma concentration maximum (Cₘₐₓ) during a nominal dosage interval (8 hours) at steady state.
Cₐᵥₑ=average concentration during steady state, AUCₛₛ/8 h
Cₘᵢₙ=measured minimum concentration during a nominal dosage interval (8 hours) at steady state
PTF%=Percent peak-trough fluctuation at steady state, 100% (Cₘₐₓ-Cₘᵢₙ)/Cₐᵥₑ

Pharmacokinetic parameters were calculated by non-compartmental or model-free methods, e.g. linear trapezoidal rule for AUC, see above and (ⁱ)

The pre-dose sample was always considered as if it had been taken simultaneously with the drug administration. If there were any deviations in sampling, the actual sampling times relative to drug administration were used if they exceeded the limit specified in Section 9.5.1.2 of this report. Missing data were not replaced or imputed in any way, i.e., they were treated as if the respective sample never had been scheduled. For the calculation of the AUC by the linear trapezoidal rule, this has the same effect as if the missing value had been estimated by linear interpolation. i Cawello W, ed.: Parameters for Compartment-free Pharmacokinetics: Standardisation of Study Design, Data Analysis and Reporting. Aachem, Shaker Verlag 1999

AUCₛₛ was regarded as unreliable and was not reported if more than two consecutive results were missing or if the concentrations were quantifiable for fewer than 5 time points. Cₘₐₓ and tₘₐₓ were regarded as unreliable if the maximum was observed preceding or following a sample with missing data. In case of multiple peaks, Cₘₐₓ and tₘₐₓ refered to the highest measured concentration even if there were earlier or later peaks. If two or more samples with the same concentrations were supplied by the analyst, tₘₐₓ referred to the earlier of these.

1. A pharmaceutical formulation comprising an immediate release component and an extended release component wherein, when said formulation is administered to a subject, said formulation provides eight-hour bioequivalence of APAP compared to two consecutive administrations of one-half equivalent dose concentrations of APAP at a four-hour interval.

2. A pharmaceutical formulation according to claim 1, wherein APAP is present in said pharmaceutical formation at a dosage strength ranging from about 325 to about 1300 mg in one or more unit dosage forms.

3. A pharmaceutical formulation according to claim 1, wherein APAP is present in the formulation at a dosage strength comprising 325, 500, or 650 mg.

4. A pharmaceutical formulation according to claim 1, wherein at least one active pharmaceutical ingredient is added to at least one of said immediate release component or said extended release component or both.

5. A pharmaceutical formulation according to claim 4, wherein the at least one active pharmaceutical ingredient is selected from the group consisting of an opioid or opioid-like compound, a non-steroidal anti-inflammatory drug (NSAID), a cyclooxygenase-II (COX-2) inhibitor, a glycine receptor antagonist, an antitussive, an expectorant, a decongestant, an antihistamine and mixtures thereof.

6. A pharmaceutical formulation according to claim 5, wherein said active pharmaceutical ingredient is an opioid or opioid-like compound.

7. A pharmaceutical formulation according to claim 6, wherein said opioid or opioid-like compound is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, and salts of any of the foregoing.

8. A pharmaceutical formulation according to claim 7, wherein said opioid or opioid-like compounds is selected from the group consisting of hydrocodone, morphine, hydromorphone, oxycodone, codeine, levorphanol, meperidine, methadone, or salts thereof, and mixtures thereof.

9. A pharmaceutical formulation according to claim 8, wherein said active pharmaceutical ingredient is a propoxyphene salt.

10. A pharmaceutical formulation according to claim 9, wherein the propoxyphene salt is selected from the group consisting of propoxyphene napsylate and propoxyphene hydrochloride.

11. A pharmaceutical formulation according to claim 9, wherein the propoxyphene salt is present in the immediate release and extended release components as a weight ratio comprising 50%IR:50%ER, 45%IR:55% ER, or 40%IR:60%ER.

12. (canceled)

13. (canceled)

14. (canceled)

15. (canceled)

16. (canceled)

17. (canceled)

18. (canceled)

19. (canceled)

20. A pharmaceutical formulation according to claim 1, wherein the immediate release component comprises APAP, at least one disintegrant, and, optionally, one or more binders.

21. (canceled)

22. (canceled)

23. (canceled)

24. A pharmaceutical formulation according to claim 1, wherein the extended release component at least one controlled release matrix polymer, and, optionally, one or more APIs.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A pharmaceutical formulation in unit dosage form comprising per dosage unit an amount of APAP within a range from about 325 to about 1300 mg of a composition comprising an immediate release component and an extended release component wherein, when said formulation is administered to a subject, said formulation provides eight-hour bioequivalence of APAP compared to two consecutive administrations of one-half equivalent dose concentrations of APAP at a four-hour interval, and wherein said formulation in unit dosage form being adapted for oral administration.

53. (canceled)

54. (canceled)

55. A pharmaceutical formulation comprising a first active ingredient which is APAP and an optional second active ingredient selected from the group consisting of one or more opiod or opiod-like compound wherein, when said formulation is administered to a subject in a single dose and at a total dosage strength of 1300 mg APAP under fasting conditions, the maximum plasma concentration (Cmax) of the APAP following administration is in the range from about 5.89_ug/mL to about 9.52_ug/mL.

56. (canceled)

57. (canceled)

58. (canceled)

59. (canceled)

60. (canceled)

61. (canceled)

62. (canceled)

63. (canceled)

64. A pharmaceutical formulation comprising a blend of (a) an immediate release component comprising acetaminophen, an optional water soluble binder, and at least one disintegrant; and (b) an extended release component comprising at least one controlled release matrix polymer, and one or more optional fillers, and one or more optional wicking agents.

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. A pharmaceutical formulation comprising a blend of an immediate release component comprising APAP and an extended release component comprising optional APAP wherein when said formulation is administered to a subject for the treatment of pain for at least 8 hours, providing said formulation is not in the form of a bi-layer tablet.

88. (canceled)

89. (canceled)

90. (canceled)

91. (canceled)

92. (canceled)

93. A method of treating pain in a subject in need of such treatment comprising administering a pharmaceutically effective amount of a pharmaceutical formulation of claim 1.

94. A method of treating pain and a disease state resulting elevated histamine levels in a subject in need of such treatment comprising administering a pharmaceutically effective amount of a pharmaceutical formulation comprising an immediate release component, an extended release component, APAP, and an antihistimine wherein, when said formulation is administered to a subject, said formulation provides eight-hour bioequivalence of APAP compared to two consecutive administrations of one-half equivalent dose concentrations of APAP at a four-hour interval.

95. A method of treating pain and symptoms of respiratory illness in a subject in need of such treatment comprising administering a pharmaceutically effective amount of a pharmaceutical formulation comprising an immediate release component, an extended release component, APAP and one or more optional decongestants, one or more optional antitussives, and one or more expectorants wherein, when said formulation is administered to a subject, said formulation provides eight-hour bioequivalence of APAP compared to two consecutive administrations of one-half equivalent dose concentrations of APAP at a four-hour interval.

96. (canceled)
